(19) United States
(12) Patent Application Publication
(10) Pub. No.: US 2016/0281177 A1
(43) Pub. Date: Sep. 29, 2016

(54) GENE SIGNATURES FOR RENAL CANCER PROGNOSIS

(71) Applicant: Myriad Genetics, Inc., Salt Lake City, UT (US)

(72) Inventors: Steven Stone, Salt Lake City, UT (US); Julia Reid, Salt Lake City, UT (US); Eric J. Askeland, Iowa City, IA (US); James A. Brown, Iowa City, IA (US)

(21) Appl. No.: 15/171,993

(22) Filed: Jun. 2, 2016

Related U.S. Application Data
(63) Continuation of application No. PCT/US2014/068628, filed on Dec. 4, 2014.

(51) Int. Cl. C12Q 1/68

(52) U.S. Cl. CPC C12Q 1/6886; C12Q 2600/118; C12Q 2600/158; C12Q 2600/112; C12Q 2600/16

(57) ABSTRACT

Biomarkers and methods using the biomarkers for prognosis of renal cancer in a patient are provided.

### RELATED APPLICATIONS

This application claims priority to Patent Cooperation Treaty International Application Number PCT/US2014/068628, filed Dec. 4, 2014 and U.S. provisional application No. 61/911,926, filed Dec. 4, 2013 the entire contents of both of which are hereby incorporated by reference.

### FIELD OF THE INVENTION

The invention generally relates to a molecular classification of disease and particularly to molecular markers for renal cancer prognosis and methods of use thereof.

### BACKGROUND OF THE INVENTION

Cancer is a major public health problem, accounting for roughly 25% of all deaths in the United States. Though many treatments have been devised for various cancers, these treatments often vary in severity of side effects. It is useful for clinicians to know how aggressive a patient's cancer is in order to determine how aggressively to treat the cancer.

For example, patients with renal cancer are often surgically treated with cytoreductive nephrectomy and optionally adjuvant therapy (e.g., immunotherapy, targeted therapy or chemotherapy), which can have severe side effects and limited efficacy. For many of these patients, however, these treatments and their associated side effects and costs are unnecessary because the cancer in these patients is not aggressive (i.e., grows slowly and is unlikely to cause mortality or significant morbidity during the patient's lifetime). In other patients the cancer is virulent (i.e., more likely to recur) and aggressive treatment is necessary to save or prolong the patient's life.

Some tools have been devised to help physicians in deciding which patients need aggressive treatment and which do not. Several clinical parameters are currently used for this purpose in various renal cancers. In clear cell renal cell cancer (ccRCC), for example, such clinical parameters include the size of the surgically-excised primary tumor, the Fuhrman nuclear grade of tumor cells, the stage of the tumor according to standard staging regimes, histological examination of the surgical margins, and evidence of lymph-vascular invasion. In recent years clinical parameters have been made more helpful through their incorporation into continuous multivariable postoperative nomograms that calculate a patient's probability of progression/recurrence for a particular cancer. As examples of nomograms useful in prostate cancer, see, e.g., Kattan et al., J. CLIN. ONCOL. (1999) 17:1499-1507 and Stephenson et al., J. CLIN. ONCOL. (2005) 23:7005-7012. Despite these advances, however, many patients are given improper cancer treatments and there is still a serious need for novel and improved tools for predicting renal cancer recurrence and metastatic progression.

### SUMMARY OF THE INVENTION

The present invention is based in part on the surprising discovery that the expression of those genes whose expression closely tracks the cell cycle (“cell-cycle progression” or “CCP” genes, or simply “cell-cycle genes” or “CCGs”, as further defined below) is particularly useful in classifying renal cancers and determining the prognosis of these cancers.

Accordingly, in a first aspect of the present invention, a method is provided for determining gene expression in a sample from a patient identified as having renal cancer, e.g., wherein said sample comprises renal cells or nucleic acids derived from renal cells. Generally, the method includes at least the following steps: (1) determining, in a sample from a patient identified as having renal cancer, the expression of a panel of genes in said sample comprising at least 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 or more cell-cycle genes (e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 genes from Table 1); and (2) providing a test value by (a) weighting the determined expression of each of a plurality of test genes selected from said panel of genes with a predefined coefficient, and (b) combining the weighted expression to provide said test value, wherein (i) at least 50%, at least 75% or at least 90% of said plurality of test genes are said at least 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 or more cell-cycle genes (e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 genes from Table 1) or (ii) said at least 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 or more cell-cycle genes (e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 genes from Table 1) are weighted to contribute at least 25%, 50%, 75%, 80%, 85%, 90%, 95%, 96%, 97%, 98%, or 99% or 100% of the test value.

In some embodiments, the step of determining the expression of the panel of genes in the tumor sample comprises measuring the amount of mRNA in the tumor sample transcribed from each of from 25 to about 200 genes; and measuring the amount of mRNA of one or more housekeeping genes in the tumor sample.

In another aspect of the present invention, a method is provided for determining the prognosis of renal cancer, which comprises (1) determining in a sample from a patient diagnosed with renal cancer, the expression of a panel of genes in said sample comprising 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 or more cell-cycle genes (e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 genes from Table 1); (2) providing a test value by (a) weighting the determined expression of each of a plurality of test genes selected from the panel of genes with a predefined coefficient, and (b) combining the weighted expression to provide the test value, wherein (i) at least 50%, at least 75% or at least 90% of said plurality of test genes are said at least 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 or more cell-cycle genes (e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 genes from Table 1) or (ii) said at least 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 or more cell-cycle genes (e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 genes from Table 1) are weighted to contribute at least 25%, 50%, 75%, 80%, 85%, 90%, 95%, 96%, 97%, 98%, or 99% or 100% of the test value, and (3) diagnosing said patient as having (a) a poor prognosis based at least in part on an increased level (e.g., overall level) of expression of the plurality of test genes or (b) a good prognosis based at least in part on no increased level of expression of the test genes.

In some embodiments of such prognosis methods, step (3) further includes comparing the test value provided in step (2) to one or more reference values and diagnosing the patient's prognosis based at least in part on such comparison. In some embodiments, the prognosis includes the patient's likelihood (e.g., increased, decreased, specific percentage probability, etc.) of cancer metastatic progression, likelihood of cancer recurrence, likelihood of cancer-specific death, or likelihood of response to the particular treatment regimen. In some subembodiments, the prognosis includes the likelihood of recurrence or the progression of metastatic cancers following surgery. In particular subembodiments, the prognosis includes the likelihood of recurrence or the progression of metastatic cancers following cytoreductive nephrectomy, including radical nephrectomy or partial nephrectomy. In other embodiments, the prognosis includes the likelihood that any recurrent or metastatic cancer will respond favorably to therapy. In certain subembodiments the prognosis includes the likelihood that any recurrent or metastatic cancer will respond favorably to a particular type of therapy, including neoadjuvant or adjuvant therapy of various types including cytokine immunotherapy, particularly with interleukin-2 or interferon-alpha, treatment with antiangiogenic agents, and/or treatment with mTOR kinase inhibitors, as well as treatment with conventional chemotherapy, e.g., vinblasine, floxuridine, 5-fluorouracil, cpecitabine, or gemcitabine. For example, in certain subembodiments the prognosis includes the likelihood that any recurrent or metastatic cancer will respond favorably to treatment with drugs such as Axitinib, Bevacizumab, Carfilzomib, Everolimus, Interfereon/Interferon type I, Interleukin-2, Lenalidomide/Revlimid, Pazopanib, Sirolimus/Rapamycin, Sorafenib, Sunitinib, Temsirolimus, Thalomid, or Tivozanib, or combinations thereof. Optionally a test value greater than the reference value is used to diagnose an increased likelihood of response to a particular type of treatment. In some embodiments the prognosis is based on the test value differing from the reference value by at least some amount (e.g., at least 0.5, 0.75, 0.85, 0.90, 0.95, 1, 2, 3, 4, 5, 6, 7, 8, 9, or 10 or more fold or standard deviations).

In some embodiments of such prognosis methods, the renal cancer for which a prognosis is to be determined is renal cell carcinoma (RCC). In some subembodiments the renal cell carcinoma is clear cell renal cell carcinoma (ccRCC), papillary renal cell carcinoma (pRCC), chromophobic renal cell carcinoma, collecting duct renal cell carcinoma (cdRCC), or unclassified renal cell carcinoma (RCC). In other embodiments of such prognosis methods, the renal cancer for which a prognosis is to be determined is transitional cell carcinoma (TCC), Wilms tumor (WT or nephroblastoma) or renal sarcoma (RS).

In some embodiments of such prognosis methods, clinical parameters are used in concert with the analysis of CCP gene expression. In particular subembodiments the clinical parameter used is selected from the group consisting of the size of the surgically-excised primary tumor, the Fuhrman nuclear grade of tumor cells, the stage of the tumor according to standard staging regimes, histological examination of the surgical margins, and evidence for lymph-vascular invasion, or combinations thereof.

In another aspect, the present invention provides a method for treating renal cancer, which comprises: determining in a tumor sample from a patient the expression of a CCP gene or a plurality of CCP genes, and recommending, prescribing or administering a particular treatment regimen. For example, in such embodiments wherein the renal cancer to be treated is ccRCC, the treatment regimen may comprise cytokine immunotherapy, particularly with interleukin-2 or interferon-alpha, treatment with antiangiogenic agents, and/or treatment with mTOR kinase inhibitors. The treatment regimen may also comprise conventional chemotherapy, e.g., vinblasine, floxuridine, 5-fluorouracil, cpecitabine, or gemcitabine. For example, for ccRCC, treatment regimens and/or targeted therapies may also involve treatment with particular drugs such as Axitinib, Bevacizumab, Carfilzomib, Everolimus, Interfereon/Interferon type I, Interleukin-2, Lenalidomide/Revlimid, Pazopanib, Sirolimus/Rapamycin, Sorafenib, Sunitinib, Temsirolimus, Thalomid, or Tivozanib, or combinations thereof. The choice of which of these therapeutic compounds or classes of compounds to administer may be based at least in part on the determined CCP gene expression alone, or the CCP score in combination with any appropriate clinical parameter. In some embodiments, a treatment regimen comprising cytokine immunotherapy, treatment with antiangiogenic agents, and/or treatment with mTOR kinase inhibitors is recommended, prescribed or administered based at least in part on the determination that the tumor sample has an increased level of CCP gene expression.

The present invention further provides a diagnostic kit for prognosing cancer in a patient identified as having renal cancer comprising, in a compartmentalized container, a plurality of oligonucleotides hybridizing to at least 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 or more test genes, wherein less than 10%, 30% or less than 40% of all of the test genes are genes not listed in Table 1; and one or more oligonucleotides hybridizing to at least one housekeeping gene. The oligonucleotides can be hybridizing probes for hybridization with the test genes under stringent conditions or primers suitable for PCR amplification of the test genes. In one embodiment, the kit consists essentially of, in a compartmentalized container, a first plurality of PCR reaction mixtures for PCR amplification of from 3 to about 300 test genes, wherein at least 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 of such test genes are listed in Table 1, and wherein each reaction mixture comprises a PCR primer pair for PCR amplifying one of the test genes; and a second plurality of PCR reaction mixtures for PCR amplification of at least one housekeeping gene. In some embodiments the kit comprises one or more computer software programs for calculating a test value derived from the expression of the test genes (e.g., the overall expression of either all test genes or some subset) and for comparing this test value to some reference value (and optionally for assigning a prognosis based on this comparison). In some embodiments such computer software is programmed to weight the test genes such that the at 25 test genes listed in Table 1 are weighted to contribute at least 50%, at least 75% or at least 85% of the test value. In some embodiments such computer software is programmed to communicate (e.g., display) that the patient has an increased likelihood of progression, recurrence, cancer-specific death, or response to a particular treatment regimen (e.g., comprising adjuvant radiation or chemotherapy) if the test value is greater than the reference value (e.g., by more than some predetermined amount). In some embodiments the computer software is programmed to communicate (e.g., display) the risk level of progression, recurrence, cancer-specific death, or response to a particular treatment regimen assignable to the patient based on the test value (e.g., based on comparison of the test value to a reference value).

The present invention also provides the use of (1) a plurality of oligonucleotides hybridizing to at least 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 test genes listed in Table 1; and (2) one or more oligonucleotides hybridizing to at least one housekeeping gene, for the manufacture of a diagnostic product for determining the expression of the test genes in a sample from a patient identified as having renal cancer to diagnose the prognosis of such cancer, wherein an increased level of the overall expression of the test genes indicates a poor prognosis, whereas if there is no increase in the overall expression of the test genes indicates a good prognosis. In some embodiments, the oligonucleotides are PCR primers suitable for PCR amplification of the test genes. In other embodiments, the oligonucleotides are probes hybridizing to the test genes under stringent conditions. In some embodiments, the plurality of oligonucleotides are probes for hybridization under stringent conditions to, or are suitable for PCR amplification of, from 3 to about 300 test genes, at least 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 of the test genes being listed in Table 1.

The present invention further provides systems related to the above methods of the invention. In one embodiment the invention provides a system for determining gene expression in a tumor sample, comprising: (1) a sample analyzer for determining the expression levels of a panel of genes in a sample comprising at least 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 test genes listed in Table 1, wherein the sample analyzer contains the sample, mRNA from the sample and expressed from the panel of genes, or cDNA synthesized from said mRNA; (2) a first computer program for (a) receiving gene expression data on said at least 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 test genes listed in Table 1, (b) weighting the determined expression of each of the test genes with a predefined coefficient, and (c) combining the weighted expression to provide a test value, wherein at least 50%, at least at least 75% of said test genes are listed in Table 1; and optionally (3) a second computer program for comparing the test value to one or more reference values each associated with a predetermined cancer prognosis. In another embodiment the invention provides a system for determining gene expression in a tumor sample, comprising: (1) a sample analyzer for determining the expression levels of a panel of genes in a sample including at least 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 test genes listed in Table 1, wherein the sample analyzer contains the sample which is from a patient identified as having renal cancer, mRNA expressed from the panel of genes in the sample, or cDNA molecules from mRNA expressed from the panel of genes in the sample; (2) a first computer program for (a) receiving gene expression data on said at least 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 test genes listed in Table 1, (b) weighting the determined expression of each of the test genes with a predefined coefficient, and (c) combining the weighted expression to provide a test value, wherein said at least 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 test genes listed in Table 1 are weighted to contribute at least 50%, at least 75% or at least 85% of the test value; and optionally (3) a second computer program for comparing the test value to one or more reference values each associated with a predetermined cancer prognosis. In some embodiments, the system further comprises a display module displaying the comparison between the test value to the one or more reference values, or displaying a result of the comparing step.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. In case of conflict, the present specification, including definitions, will control. In addition, the materials, methods, and examples are illustrative only and not intended to be limiting.

Other features and advantages of the invention will be apparent from the following Detailed Description, and from the Claims.

### BRIEF DESCRIPTION OF THE DRAWINGS

FIG. 1 illustrates the distribution of CCP scores among eligible patients.

FIG. 2 depicts time versus CCP score for all patients with metastatic cancer.

FIG. 3 is a Kaplan-Meier estimate plot with 95% confidence bounds for all patients with metastatic cancer.

FIG. 4 illustrates an example of a computer system useful in certain aspects and embodiments of the invention.

FIG. 5 is a flowchart illustrating an example of a computer-implemented method of the invention.

### DETAILED DESCRIPTION OF THE INVENTION

### I. Determining Cell-Cycle Progression Gene Expression

The present invention is based in part on the discovery that genes whose expression closely tracks the cell cycle (“cell-cycle progression genes,” “CCP genes,” “cell-cycle genes,” or sometimes “CCGs”) are particularly powerful genes for classifying and prognosing renal cancers.

“Cell-cycle gene” and “CCG” herein refer to a gene whose expression level closely tracks the progression of the cell through the cell-cycle. See, e.g., Whitfield et al., MOL. BIOL. CELL (2002) 13:1977-2000. The term “cell-cycle progression” or “CCP” will also be used in this application and will generally be interchangeable with CCG (i.e., a CCP gene is a CCG; a CCP score is a CCG score). More specifically, CCP genes show periodic increases and decreases in expression that coincide with certain phases of the cell cycle—e.g., STK15 and PLK show peak expression at G2/M. Id. Often CCP genes have clear, recognized cell-cycle related function—e.g., in DNA synthesis or repair, in chromosome condensation, in cell-division, etc. However, some CCP genes have expression levels that track the cell-cycle without having an obvious, direct role in the cell-cycle—e.g., UBE2S encodes a ubiquitin-conjugating enzyme, yet its expression closely tracks the cell-cycle. Thirty-one (31) CCP genes useful according to the present disclosure are listed in Table 1. A more complete discussion of CCP genes can be found in International Application No. PCT/US2010/020397 (pub. no. WO/2010/080933) (see, e.g., Table 1 in WO/2010/080933), U.S. utility application Ser. No. 13/177,887 (pub. no. US20120041274), International Application No. PCT/US2011/043228 (pub. no. WO/2012/006447), and U.S. utility application Ser. No. 13/178,380 (pub. no. US20120053253), the contents of which are hereby incorporated by reference in their entirety.

TABLE 1
Thirty-One Cell Cycle Progression Genes

| Gene | Entrez | Example ABI | Example RefSeq |
|---|---|---|---|
| Symbol | GeneID | Assay ID | Accession Nos. |
| ASF1B | 55723 | Hs00216780_m1 | NM_018154.2 |
| ASPM | 259266 | Hs00411505_m1 | NM_018136.4 |
| BIRC5 | 332 | Hs00153353_m1; | NM_001012271.1; |
| | | Hs03043576_m1 | NM_001012270.1; |
| | | | NM_001168.2 |
| BUB1B | 701 | Hs01084828_m1 | NM_001211.5 |
| C18orf24 | 220134 | Hs00536843_m1 | NM_145060.3; |
| | | | NM_001039535.2 |
| CDC2 | 983 | Hs00364293_m1 | NM_033379.3; |
| | | | NM_001130829.1; |
| | | | NM_001786.3 |
| CDC20 | 991 | Hs03004916_g1 | NM_001255.2 |
| CDCA3 | 83461 | Hs00229905_m1 | NM_031299.4 |
| CDCA8 | 55143 | Hs00983655_m1 | NM_018101.2 |
| CDKN3 | 1033 | Hs00193192_m1 | NM_001130851.1; |
| | | | NM_005192.3 |
| CENPF | 1063 | Hs00193201_m1 | NM_016343.3 |
| CENPM | 79019 | Hs00608780_m1 | NM_024053.3 |
| CEP55 | 55165 | Hs00216688_m1 | NM_018131.4; |
| | | | NM_001127182.1 |
| DLGAP5 | 9787 | Hs00207323_m1 | NM_014750.3 |
| DTL | 51514 | Hs00978565_m1 | NM_016448.2 |
| FOXM1 | 2305 | Hs01073586_m1 | NM_202003.1; |
| | | | NM_202002.1; |
| | | | NM_021953.2 |
| KIAA0101 | 9768 | Hs00207134_m1 | NM_014736.4 |
| KIF11 | 3832 | Hs00189698_m1 | NM_004523.3 |
| KIF20A | 10112 | Hs00993573_m1 | NM_005733.2 |
| MCM10 | 55388 | Hs00960349_m1 | NM_018518.3; |
| | | | NM_182751.1 |
| NUSAP1 | 51203 | Hs01006195_m1 | NM_018454.6; |
| | | | NM_001129897.1; |
| | | | NM_016359.3 |
| ORC6L | 23594 | Hs00204876_m1 | NM_014321.2 |
| PBK | 55872 | Hs00218544_m1 | NM_018492.2 |
| PLK1 | 5347 | Hs00153444_m1 | NM_005030.3 |
| PRC1 | 9055 | Hs00187740_m1 | NM_199413.1; |
| | | | NM_199414.1; |
| | | | NM_003981.2 |
| PTTG1 | 9232 | Hs00851754_u1 | NM_004219.2 |
| RAD51 | 5888 | Hs00153418_m1 | NM_133487.2; |
| | | | NM_002875.3 |
| RAD54L | 8438 | Hs00269177_m1 | NM_001142548.1; |
| | | | NM_003579.3 |
| RRM2 | 6241 | Hs00357247_g1 | NM_001034.2 |
| TK1 | 7083 | Hs01062125_m1 | NM_003258.4 |
| TOP2A | 7153 | Hs00172214_m1 | NM_001067.2 |

Accordingly, in a first aspect of the present invention, a method is provided for determining gene expression in a tumor sample from a patient identified as having renal cancer. Generally, the method includes at least the following steps: (1) obtaining a tumor sample from a patient (e.g., one identified as having renal cancer); (2) determining the expression of a panel of genes in the tumor sample including at least 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 or more cell-cycle genes (e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 genes from Table 1); and (3) providing a test value by (a) weighting the determined expression of each of a plurality of test genes selected from said panel of genes with a predefined coefficient, and (b) combining the weighted expression to provide said test value, wherein at least 20%, at least 50%, at least 60%, at least 70%, at least 75%, at least 80%, at least 85%, at least 90%, at least 95% or at least 96, 97, 98 or 99% of said plurality of test genes are cell-cycle genes. In some embodiments the test genes are weighted such that the cell-cycle genes are weighted to contribute at least 50%, at least 55%, at least 60%, at least 65%, at least 75%, at least 80%, at least 85%, at least 90%, at least 95%, at least 99% or 100% of the test value. In some embodiments 20%, 25%, 30%, 35%, 40%, 45%, 50%, 55%, 60%, 65%, 75%, 80%, 85%, 90%, 95%, or at least 99% or 100% of the plurality of test genes are cell-cycle genes.

Gene expression can be determined either at the RNA level (i.e., mRNA or noncoding RNA (ncRNA)) (e.g., miRNA, tRNA, rRNA, snoRNA, siRNA and piRNA) or at the protein level. Measuring gene expression at the mRNA level includes measuring levels of cDNA corresponding to mRNA. Levels of proteins in a tumor sample can be determined by any known techniques in the art, e.g., HPLC, mass spectrometry, or using antibodies specific to selected proteins (e.g., IHC, ELISA, etc.).

In preferred embodiments, the amount of RNA transcribed from the panel of genes including test genes is measured in the tumor sample. In addition, the amount of RNA transcribed from one or more housekeeping genes in the tumor sample is also measured, and is used to normalize or calibrate the expression of the test genes. The terms “normalizing genes” and “housekeeping genes” are defined herein below.

In any embodiment of the invention involving a “plurality of test genes,” the plurality of test genes may include at least 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 or more cell-cycle genes (e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 genes from Table 1), which may constitute at least 50%, 75% or 80% of the plurality of test genes, and preferably 100% of the plurality of test genes. In some embodiments, the plurality of test genes includes at least 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 or more cell-cycle genes (e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 genes from Table 1), which may constitute at least 20%, 25%, 30%, 40%, 50%, 60%, 70%, 75%, 80% or 90% of the plurality of test genes, and preferably 100% of the plurality of test genes. As will be clear from the context of this document, a panel of genes is also a plurality of genes. Typically these genes are assayed together in one or more samples from a patient.

As will be apparent to a skilled artisan apprised of the present invention and the disclosure herein, “tumor sample” means any biological sample containing one or more tumor cells, or one or more tumor derived RNAs or proteins, and obtained from a cancer patient. For example, a tissue sample obtained from a tumor tissue of a cancer patient is a useful tumor sample in the present invention. The tissue sample can be a formalin fixed, paraffin embedded (FFPE) sample, or fresh frozen sample, and preferably contain largely tumor cells. A single malignant cell from a cancer patient's tumor is also a useful tumor sample. Such a malignant cell can be obtained directly from the patient's tumor, or purified from the patient's bodily fluid (e.g., blood, urine). Thus, a bodily fluid such as blood, urine, sputum and saliva containing one or tumor cells, or tumor-derived RNA or proteins, can also be useful as a tumor sample for purposes of practicing the present invention.

Those skilled in the art are familiar with various techniques for determining the status of a gene or protein in a tissue or cell sample including, but not limited to, microarray analysis (e.g., for assaying mRNA or microRNA expression, copy number, etc.), quantitative real-time PCR™ (“qRT-PCR™”, e.g., TaqMan™), immunoanalysis (e.g., ELISA, immunohistochemistry), etc. The activity level of a polypeptide encoded by a gene may be used in much the same way as the expression level of the gene or polypeptide. Often higher activity levels indicate higher expression levels and while lower activity levels indicate lower expression levels. Thus, in some embodiments, the invention provides any of the methods discussed above, wherein the activity level of a polypeptide encoded by the CCG is determined rather than or in addition to the expression level of the CCG. Those skilled in the art are familiar with techniques for measuring the activity of various such proteins, including those encoded by the genes listed in Tables 1 & 2. The methods of the invention may be practiced independent of the particular technique used.

In some embodiments, the expression of one or more normalizing (often called “housekeeping” or “housekeeper”) genes is also obtained for use in normalizing the expression of test genes. As used herein, “normalizing genes” referred to the genes whose expression is used to calibrate or normalize the measured expression of the gene of interest (e.g., test genes). Importantly, the expression of normalizing genes should be independent of cancer outcome/prognosis, and the expression of the normalizing genes is very similar among all the tumor samples. The normalization ensures accurate comparison of expression of a test gene between different samples. For this purpose, housekeeping genes known in the art can be used. Housekeeping genes are well known in the art, with examples including, but are not limited to, GUSB (glucuronidase, beta), HMBS (hydroxymethylbilane synthase), SDHA (succinate dehydrogenase complex, subunit A, flavoprotein), UBC (ubiquitin C) and YWHAZ (tyrosine 3-monooxygenase/tryptophan 5-monooxygenase activation protein, zeta polypeptide). One or more housekeeping genes can be used. In some embodiments, at least 2, 5, 10 or 15 housekeeping genes are used to provide a combined normalizing gene set. The amount of gene expression of such normalizing genes can be averaged, combined together by straight additions or by a defined algorithm. Some examples of particularly useful housekeeper genes for use in the methods and compositions of the invention include those listed in Table 2 below.

TABLE 2

| Gene | Entrez | Applied Biosystems | |
|---|---|---|---|
| Symbol | GeneID | Assay ID | RefSeq Accession Nos. |
| CLTC* | 1213 | Hs00191535_m1 | NM_004859.3 |
| GUSB | 2990 | Hs99999908_m1 | NM_000181.2 |
| HMBS | 3145 | Hs00609297_m1 | NM_000190.3 |
| MMADHC* | 27249 | Hs00739517_g1 | NM_015702.2 |
| MRFAP1* | 93621 | Hs00738144_g1 | NM_033296.1 |
| PPP2CA* | 5515 | Hs00427259_m1 | NM_002715.2 |
| PSMA1* | 5682 | Hs00267631_m1 | |
| PSMC1* | 5700 | Hs02386942_g1 | NM_002802.2 |
| RPL13A* | 23521 | Hs03043885_g1 | NM_012423.2 |
| RPL37* | 6167 | Hs02340038_g1 | NM_000997.4 |
| RPL38* | 6169 | Hs00605263_g1 | NM_000999.3 |
| RPL4* | 6124 | Hs03044647_g1 | NM_000968.2 |
| RPL8* | 6132 | Hs00361285_g1 | NM_033301.1; |
| | | | NM_000973.3 |
| RPS29* | 6235 | Hs03004310_g1 | NM_001030001.1; |
| | | | NM_001032.3 |
| SDHA | 6389 | Hs00188166_m1 | NM_004168.2 |
| SLC25A3* | 6515 | Hs00358082_m1 | NM_213611.1; |
| | | | NM_002635.2; |
| | | | NM_005888.2 |
| TXNL1* | 9352 | Hs00355488_m1 | NR_024546.1; |
| | | | NM_004786.2 |
| UBA52* | 7311 | Hs03004332_g1 | NM_001033930.1; |
| | | | NM_003333.3 |
| UBC | 7316 | Hs00824723_m1 | NM_021009.4 |
| YWHAZ | 7534 | Hs00237047_m1 | NM_003406.3 |

*Subset of 15 housekeeping genes used in, e.g., EXAMPLE 1.

In the case of measuring RNA levels for the genes, one convenient and sensitive approach is real-time quantitative PCR™ (qPCR) assay, following a reverse transcription reaction. Typically, a cycle threshold (Cₜ) is determined for each test gene and each normalizing gene, i.e., the number of cycles at which the fluorescence from a qPCR reaction above background is detectable.

The overall expression of the one or more normalizing genes can be represented by a “normalizing value” which can be generated by combining the expression of all normalizing genes, either weighted equally (straight addition or averaging) or by different predefined coefficients. For example, in a simplest manner, the normalizing value C_{tH} can be the cycle threshold (Cₜ) of one single normalizing gene, or an average of the Cₜ values of 2 or more, 5 or more, 10 or more, or 15 or more normalizing genes, in which case, the predefined coefficient is 1/N, where N is the total number of normalizing genes used. Thus, C_{tH}=(C_{tH1}+C_{tH2}+ . . . C_{tHn})/N. As will be apparent to skilled artisans, depending on the normalizing genes used, and the weight desired to be given to each normalizing gene, any coefficients (from 0/N to N/N) can be given to the normalizing genes in weighting the expression of such normalizing genes. That is, C_{tH}=xC_{tH1}+yC_{tH2}+ . . . zC_{tHn}, wherein x+y+ . . . +z=1.

As discussed above, the methods of the invention generally involve determining the level of expression of a panel of CCP genes. With modern high-throughput techniques, it is often possible to determine the expression level of tens, hundreds or thousands of genes. Indeed, it is possible to determine the level of expression of the entire transcriptome (i.e., each transcribed sequence in the genome). Once such a global assay has been performed, one may then informatically analyze one or more subsets of transcripts (i.e., panels or, as often used herein, pluralities of test genes). After measuring the expression of hundreds or thousands of transcripts in a sample, for example, one may analyze (e.g., informatically) the expression of a panel or plurality of test genes comprising primarily CCP genes according to the present invention by combining the expression level values of the individual test genes to obtain a test value.

As will be apparent to a skilled artisan, the test value provided in the present invention represents the overall expression level of the plurality of test genes composed substantially of cell-cycle progression genes. In one embodiment, to provide a test value in the methods of the invention, the normalized expression for a test gene can be obtained by normalizing the measured Cₜ for the test gene against the C_{tH}, i.e., ΔCₜ₁=(Cₜ₁−C_{tH}). Thus, the test value representing the overall expression of the plurality of test genes can be provided by combining the normalized expression of all test genes, either by straight addition or averaging (i.e., weighted equally) or by a different predefined coefficient. For example, the simplest approach is averaging the normalized expression of all test genes: test value=(ΔCₜ₁+ΔCₜ₂+ . . . +ΔCₜₙ)/n. As will be apparent to skilled artisans, depending on the test genes used, different weight can also be given to different test genes in the present invention. For example, in some embodiments described above, the plurality of test genes comprises at least 2 CCP genes, and the combined weight given to the at least 2 CCP genes is at least 10%, 15%, 20%, 25%, 30%, 35%, 40%, 45%, 50%, 55%, 60%, 65%, 70%, 75%, 80%, 85%, 90%, 95%, 96%, 97%, 98%, or 99% or 100% of the total weight given to all of said plurality of test genes. That is, test value=xΔCₜ₁+yΔCₜ₂+ . . . +zΔCₜₙ, wherein ΔCₜ₁ and ΔCₜ₂ represent the gene expression of the 2 CCP genes, respectively, and (x+y)/(x+y+ . . . +z) is at least 10%, 15%, 20%, 25%, 30%, 35%, 40%, 45%, 50%, 55%, 60%, 65%, 70%, 75%, 80%, 85%, 90%, 95%, 96%, 97%, 98%, or 99% or 100%.

In each case where this document discloses using the expression of a plurality of genes (e.g., “determining [in a tumor sample from the patient] the expression of a plurality of test genes” or “correlating increased expression of said plurality of test genes to an increased likelihood of recurrence”), this includes in some embodiments using a test value representing, corresponding to or derived or calculated from the overall expression of this plurality of genes (e.g., “determining [in a tumor sample from the patient] a test value representing the expression of a plurality of test genes” or “correlating an increased test value [or a test value above some reference value](optionally representing the expression of said plurality of test genes) to an increased likelihood of response”).

In some embodiments, many CCGs are very good surrogates for each other. Thus any CCG (or panel of CCGs) can be used in the various embodiments of the invention. In other embodiments of the invention, optimized CCGs are used. One way of assessing whether particular CCGs will serve well in the methods and compositions of the invention is by assessing their correlation with the mean expression of CCGs (e.g., all known CCGs, a specific set of CCGs, etc.). Those CCGs that correlate particularly well with the mean are expected to perform well in assays of the invention, e.g., because these will reduce noise in the assay.

### II. Cancer Prognosis

It has been surprisingly discovered that in selected renal cancers, the expression of cell-cycle genes in tumor cells can accurately predict the degree of aggressiveness of the cancer and risk of recurrence or metastatic progression after treatment (e.g., surgical removal of cancer tissue through cytoreductive nephrectomy, adjuvant therapy, etc.). Thus, the above-described method of determining cell-cycle gene expression can be applied in the prognosis and treatment of such cancers.

Generally, a method is provided for prognosing renal cancer in patients, which comprises measuring the expression of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 or more cell-cycle genes (e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 genes from Table 1) in one or more patient samples and diagnosing (a) a poor prognosis in a patient in whose sample expression of said cell-cycle genes exceeds some reference or (b) a good prognosis in a patient in whose sample expression of said cell-cycle genes does not exceed some reference. The expression can be determined in accordance with the methods described above.

The present disclosure provides a related method for prognosing renal cancer, which comprises determining in a tumor sample from a patient diagnosed of renal cancer, the expression of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 or more cell-cycle genes (e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 genes from Table 1), wherein high expression (or increased expression or overexpression) of the cell-cycle genes indicates a poor prognosis or an increased likelihood of recurrence or metastatic progression of cancer in the patient. The expression can be determined in accordance with the method described above. In some embodiments, the method comprises at least one of the following steps: (a) correlating high expression (or increased expression or overexpression) of the cell-cycle genes to a poor prognosis or an increased likelihood of recurrence or metastatic progression of cancer in the patient; (b) concluding that the patient has a poor prognosis or an increased likelihood of recurrence or metastatic progression of cancer based at least in part on high expression (or increased expression or overexpression) of the cell-cycle genes; or (c) communicating that the patient has a poor prognosis or an increased likelihood of recurrence or metastatic progression of cancer based at least in part on high expression (or increased expression or overexpression) of the cell-cycle genes.

In each embodiment described in this document involving correlating a particular assay or analysis output (e.g., high CCP expression, test value incorporating CCP expression greater than some reference value, etc.) to some likelihood (e.g., increased, not increased, decreased, etc.) of some clinical event or outcome (e.g., recurrence, metastatic progression, cancer-specific death, etc.), such correlating may comprise assigning a risk or likelihood of the clinical event or outcome occurring based at least in part on the particular assay or analysis output. In some embodiments, such risk is a percentage probability of the event or outcome occurring. In some embodiments, the patient is assigned to a risk group (e.g., low risk, intermediate risk, high risk, etc.). In some embodiments “low risk” is any percentage probability below 5%, 10%, 15%, 20%, 25%, 30%, 35%, 40%, 45%, or 50%. In some embodiments “intermediate risk” is any percentage probability above 5%, 10%, 15%, 20%, 25%, 30%, 35%, 40%, 45%, or 50% and below 15%, 20%, 25%, 30%, 35%, 40%, 45%, 50%, 55%, 60%, 65%, 70%, or 75%. In some embodiments “high risk” is any percentage probability above 25%, 30%, 35%, 40%, 45%, 50%, 55%, 60%, 65%, 70%, 75%, 80%, 85%, 90%, 95%, or 99%.

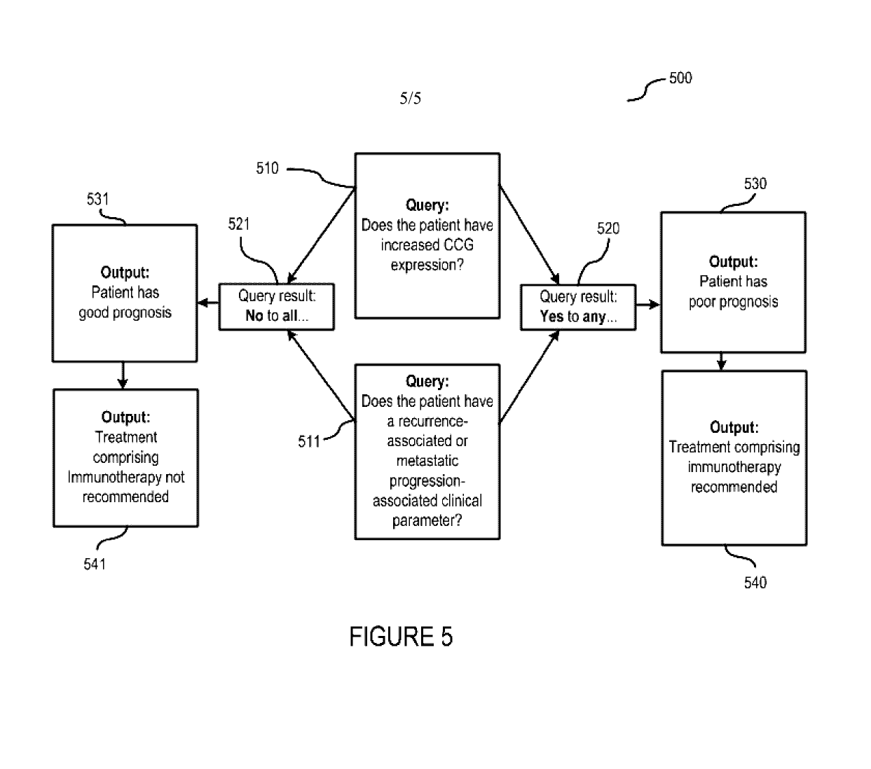

As used herein, “communicating” a particular piece of information means to make such information known to another person or transfer such information to a thing (e.g., a computer). In some methods of the invention, a patient's prognosis or risk of recurrence is communicated. In some embodiments, the information used to arrive at such a prognosis or risk prediction (e.g., expression levels of a panel of biomarkers comprising a plurality of CCGs, clinical or pathologic factors, etc.) is communicated. This communication may be auditory (e.g., verbal), visual (e.g., written), electronic (e.g., data transferred from one computer system to another), etc. In some embodiments, communicating a cancer classification comprises generating a report that communicates the cancer classification. In some embodiments the report is a paper report, an auditory report, or an electronic record. In some embodiments the report is displayed and/or stored on a computing device (e.g., handheld device, desktop computer, smart device, website, etc.). In some embodiments the cancer classification is communicated to a physician (e.g., a report communicating the classification is provided to the physician). In some embodiments the cancer classification is communicated to a patient (e.g., a report communicating the classification is provided to the patient). Communicating a cancer classification can also be accomplished by transferring information (e.g., data) embodying the classification to a server computer and allowing an intermediary or end-user to access such information (e.g., by viewing the information as displayed from the server, by downloading the information in the form of one or more files transferred from the server to the intermediary or end-user's device, etc.).

Wherever an embodiment of the invention comprises concluding some fact (e.g., a patient's prognosis or a patient's likelihood of recurrence), this may include a computer program concluding such fact, typically after performing an algorithm that applies information on CCG status in a patient sample and/or the presence or absence of clinical variables associated with cancer recurrence or metastatic progression (e.g., as shown in FIG. 5).

In some embodiments, the prognosis method includes (1) obtaining a tumor sample from a patient identified as having renal cancer; (2) determining the expression of a panel of genes in the tumor sample including at least 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 or more cell-cycle genes (e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 genes from Table 1); and (3) providing a test value by (a) weighting the determined expression of each of a plurality of test genes selected from the panel of genes with a predefined coefficient, and (b) combining the weighted expression to provide said test value, wherein at least 20%, 50%, at least 75% or at least 90% of said plurality of test genes are cell-cycle genes (e.g., genes from Table 1), and wherein high expression (or increased expression or overexpression) of the plurality of test genes indicates a poor prognosis or an increased likelihood of cancer recurrence or metastatic progression. In some embodiments, the method comprises at least one of the following steps: (a) correlating high expression (or increased expression or overexpression) of the plurality of test genes to a poor prognosis or an increased likelihood of recurrence or metastatic progression of cancer in the patient; (b) concluding that the patient has a poor prognosis or an increased likelihood of recurrence or metastatic progression of cancer based at least in part on high expression (or increased expression or overexpression) of the plurality of test genes; or (c) communicating that the patient has a poor prognosis or an increased likelihood of recurrence or metastatic progression of cancer based at least in part on high expression (or increased expression or overexpression) of the plurality of test genes.

In some embodiments, the expression levels measured in a sample are used to derive or calculate a value or score. This value may be derived solely from the expression levels of the test genes (e.g., a CCG score) or optionally derived from a combination of the expression value/score with other components (e.g., size of the excised tumor, Fuhrman nuclear score, status of surgical margins, and evidence of lymph-vascular invasion, etc.) to give a more comprehensive value/score. Thus, in every case where an embodiment of the invention described herein involves determining the status of a biomarker (e.g., RNA expression levels of a CCG), related embodiments involve deriving or calculating a value or score from the measured status (e.g., expression score).

In some such embodiments, multiple scores (e.g., CCG, tumor size, Fuhrman nuclear score, and evidence of lymph-vascular invasion) can be combined into a more comprehensive score. Single component (e.g., CCG) or combined test scores for a particular patient can be compared to single component or combined scores for reference populations as described below, with differences between test and reference scores being correlated to or indicative of some clinical feature. Thus, in some embodiments the invention provides a method of determining a cancer patient's prognosis comprising (1) obtaining the measured expression levels of a plurality of genes comprising a plurality of CCGs in a sample from the patient (e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 more genes from Table 1), (2) calculating a test value from these measured expression levels, (3) comparing said test value to a reference value calculated from measured expression levels of the plurality of genes in a reference population of patients, and (4)(a) correlating a test value greater than the reference value to a poor prognosis or (4)(b) correlating a test value equal to or less than the reference value to a good prognosis.

In some such embodiments the test value is calculated by averaging the measured expression of the plurality of genes (as discussed below). In some embodiments the test value is calculated by weighting each of the plurality of genes in a particular way.

In some embodiments the plurality of CCGs are weighted such that they contribute at least some proportion of the test value (e.g., 10%, 20%, 30%, 40%, 50%, 60%, 70%, 80%, 90%, 95%, 99%, 100%). In some embodiments each member of the plurality of CCGs is weighted such that not all are given equal weight (e.g., FOXM1 weighted to contribute more to the test value than one, some or all other genes or CCGs).

In some embodiments, the test value derived or calculated from a particular CCG (e.g., FOXM1) or from the overall expression of the plurality of test genes (e.g., CCGs) is compared to one or more reference values (or index values), and the test value is optionally correlated to prognosis, risk of cancer recurrence, risk of metastatic cancer progression, or risk of cancer-specific death if it differs from the index value.

For example, the index value may be derived or calculated from the gene expression levels found in a normal sample obtained from the patient of interest, in which case a test value (derived or calculated from an expression level in the tumor sample) significantly higher than this index value would indicate, e.g., a poor prognosis or increased likelihood of cancer recurrence, increased likelihood of metastatic cancer progression, increased likelihood of cancer-specific death, or a need for aggressive treatment. In some embodiments the test value is deemed “greater than” the reference value (e.g., the threshold index value), and thus correlated to an increased likelihood of response to treatment comprising adjuvant therapy, including cytokine immunotherapy, targeted therapy, or conventional chemotherapy, or combinations thereof, if the test value exceeds the reference value by at least some amount (e.g., at least 0.5, 0.75, 0.85, 0.90, 0.95, 1, 2, 3, 4, 5, 6, 7, 8, 9, or 10 or more fold or standard deviations).

Alternatively, the index value may be derived or calculated from the average expression level for a set of individuals from a diverse cancer population or a subset of the population. For example, one may determine the average expression level of a gene or gene panel in a random sampling of patients with renal cancer. This average expression level may be termed the “threshold index value,” with patients having CCG expression higher than this value expected to have a poorer prognosis than those having expression lower than this value.

Alternatively the index value may represent the average expression level of a particular gene marker or plurality of markers in a plurality of training patients (e.g., renal cancer patients) with similar outcomes whose clinical and follow-up data are available and sufficient to define and categorize the patients by disease outcome, e.g., recurrence, metastatic progression or prognosis. See, e.g., Examples, infra. For example, a “good prognosis index value” can be generated from a plurality of training cancer patients characterized as having “good outcome”, e.g., those who have not had cancer recurrence five years (or ten years or more) after initial treatment, or who have not had metastatic progression of their cancer five years (or ten years or more) after initial diagnosis. A “poor prognosis index value” can be generated from a plurality of training cancer patients defined as having “poor outcome”, e.g., those who have had cancer recurrence within five years (or ten years, etc.) after initial treatment, or who have had metastatic progression of their cancer within five years (or ten years, etc.) after initial diagnosis. Thus, a good prognosis index value of a particular gene may represent the average level of expression of the particular gene in patients having a “good outcome,” whereas a poor prognosis index value of a particular gene represents the average level of expression of the particular gene in patients having a “poor outcome.”

Thus one aspect of the invention provides a method of classifying cancer comprising determining the status of a panel of genes comprising at least 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 or more cell-cycle genes (e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 genes from Table 1), in tissue or cell sample, particularly a tumor sample, from a patient, wherein an abnormal status indicates a negative cancer classification. As used herein, “determining the status” of a gene refers to determining the presence, absence, or extent/level of some physical, chemical, or genetic characteristic of the gene or its expression product(s). Such characteristics include, but are not limited to, expression levels, activity levels, mutations, copy number, methylation status, etc.

In the context of CCGs as used to determine risk of cancer recurrence or metastatic progression or need for aggressive treatment, particularly useful characteristics include expression levels (e.g., mRNA or protein levels) and activity levels. Characteristics may be assayed directly (e.g., by assaying a CCG's expression level) or determined indirectly (e.g., assaying the level of a gene or genes whose expression level is correlated to the expression level of the CCG). Thus some embodiments of the invention provide a method of classifying cancer comprising determining the expression level, particularly mRNA level of a panel of genes comprising at least 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 or more cell-cycle genes (e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 genes from Table 1), in a tumor sample, wherein high expression (or increased expression or overexpression) indicates a negative cancer classification, an increased risk of cancer recurrence, an increased risk of metastatic progression, or a need for aggressive treatment. In some embodiments, the method comprises at least one of the following steps: (a) correlating high expression (or increased expression or overexpression) of the panel of genes to a negative cancer classification, an increased risk of cancer recurrence or metastatic progression, or a need for aggressive treatment; (b) concluding that the patient has a negative cancer classification, an increased risk of cancer recurrence, an increased risk of metastatic cancer progression, or a need for aggressive treatment based at least in part on high expression (or increased expression or overexpression) of the panel of genes; or (c) communicating that the patient has a negative cancer classification, an increased risk of cancer recurrence, an increased risk of metastatic cancer progression, or a need for aggressive treatment based at least in part on high expression (or increased expression or overexpression) of the panel of genes.

“Abnormal status” means a marker's status in a particular sample differs from the status generally found in average samples (e.g., healthy samples or average diseased samples). Examples include mutated, elevated, decreased, present, absent, etc. An “elevated status” means that one or more of the above characteristics (e.g., expression or mRNA level) is higher than normal levels. Generally this means an increase in the characteristic (e.g., expression or mRNA level) as compared to an index value. Conversely a “low status” means that one or more of the above characteristics (e.g., gene expression or mRNA level) is lower than normal levels. Generally this means a decrease in the characteristic (e.g., expression) as compared to an index value. In this context, a “negative status” generally means the characteristic is absent or undetectable. For example, FOXM1 status is negative if FOXM1 nucleic acid and/or protein is absent or undetectable in a sample. However, negative FOXM1 status also includes a mutation or copy number reduction in FOXM1.

In some embodiments of the invention the methods comprise determining the expression of 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 or more cell-cycle genes (e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 genes from Table 1) and, if this expression is “increased,” the patient has a poor prognosis. In the context of the invention, “increased” expression of a CCG means the patient's expression level is either elevated over a normal index value or a threshold index (e.g., by at least some threshold amount) or closer to the “poor prognosis index value” than to the “good prognosis index value.”

Thus, when the determined level of expression of a relevant gene marker is closer to the good prognosis index value of the gene than to the poor prognosis index value of the gene, then it can be concluded that the patient is more likely to have a good prognosis, i.e., a low (or no increased) likelihood of cancer recurrence and metastatic progression. On the other hand, if the determined level of expression of a relevant gene marker is closer to the poor prognosis index value of the gene than to the good prognosis index value of the gene, then it can be concluded that the patient is more likely to have a poor prognosis, i.e., an increased likelihood of cancer recurrence or metastatic progression.

Alternatively index values may be determined thusly: In order to assign patients to risk groups, a threshold value will be set for the cell cycle mean. The optimal threshold value is selected based on the receiver operating characteristic (ROC) curve, which plots sensitivity vs (1−specificity). For each increment of the cell cycle mean, the sensitivity and specificity of the test is calculated using that value as a threshold. The actual threshold will be the value that optimizes these metrics according to the artisans requirements (e.g., what degree of sensitivity or specificity is desired, etc.).

Panels of CCGs (e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 more genes from Table 1) can predict prognosis. Those skilled in the art are familiar with various ways of determining the expression of a panel of genes (i.e., a plurality of genes). One may determine the expression of a panel of genes by determining the average expression level (normalized or absolute) of all panel genes in a sample obtained from a particular patient (either throughout the sample or in a subset of cells from the sample or in a single cell). Increased expression in this context will mean the average expression is higher than the average expression level of these genes in normal patients (or higher than some index value that has been determined to represent the average expression level in a reference population such as patients with the same cancer). Alternatively, one may determine the expression of a panel of genes by determining the average expression level (normalized or absolute) of at least a certain number (e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 more genes from Table 1) or at least a certain proportion (e.g., 10%, 20%, 30%, 40%, 50%, 60%, 70%, 80%, 90%, 95%, 99%, 100%) of the genes in the panel. Alternatively, one may determine the expression of a panel of genes by determining the absolute copy number of the mRNA (or protein) of all the genes in the panel and either total or average these across the genes.

As used herein, “classifying a cancer” and “cancer classification” refer to determining one or more clinically-relevant features of a cancer and/or determining a particular prognosis of a patient having said cancer. Thus “classifying a cancer” includes, but is not limited to: (i) evaluating metastatic potential, potential to metastasize to specific organs, risk of recurrence, and/or course of the tumor; (ii) evaluating tumor stage; (iii) determining patient prognosis in the absence of treatment of the cancer; (iv) determining prognosis of patient response (e.g., tumor shrinkage or progression-free survival) to treatment (e.g., surgery to excise tumor, adjuvant therapy, including immunotherapy, targeted therapy, or conventional chemotherapy, etc.); (v) diagnosis of actual patient response to current and/or past treatment; (vi) determining a preferred course of treatment for the patient; (vii) prognosis for patient relapse after treatment (either treatment in general or some particular treatment); (viii) prognosis of patient life expectancy (e.g., prognosis for overall survival), etc.

Thus, a “negative classification” means an unfavorable clinical feature of the cancer (e.g., a poor prognosis). Examples include (i) an increased metastatic potential, potential to metastasize to specific organs, and/or risk of recurrence; (ii) an advanced tumor stage; (iii) a poor patient prognosis in the absence of treatment of the cancer; (iv) a poor prognosis of patient response (e.g., tumor shrinkage or progression-free survival) to a particular treatment (e.g., surgery to excise tumor, adjuvant therapy, including immunotherapy, targeted therapy, or conventional chemotherapy, etc.); (v) a poor prognosis for patient relapse after treatment (either treatment in general or some particular treatment); (vi) a poor prognosis of patient life expectancy (e.g., prognosis for overall survival), etc. In some embodiments a recurrence-associated or metastatic progression-associated clinical parameter (or a high nomogram score) and increased expression of a CCG indicate a negative classification in cancer (e.g., increased likelihood of recurrence or progression).

A patient in whose sample CCP expression, score or value is high has an increased likelihood of recurrence after treatment (e.g., the cancer cells not killed or removed by the treatment will quickly grow back). Such a patient also has an increased likelihood of cancer progression for more rapid progression (e.g., the rapidly proliferating cells will cause any tumor to grow quickly, gain in virulence, and/or metastasize). Such a patient may also require a relatively more aggressive treatment. Thus, in some embodiments the invention provides a method of classifying cancer comprising determining the status of a panel of genes comprising at least 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 or more cell-cycle genes (e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 genes from Table 1), wherein an abnormal status indicates an increased likelihood of recurrence or metastatic progression. In some embodiments, the method comprises at least one of the following steps: (a) correlating abnormal status of the panel of genes to an increased likelihood of recurrence or metastatic progression; (b) concluding that the patient has an increased likelihood of recurrence or metastatic progression based at least in part on abnormal status of the panel of genes; or (c) communicating that the patient has an increased likelihood of recurrence or metastatic progression based at least in part on abnormal status of the panel of genes. As discussed above, in some embodiments the status to be determined is CCG expression levels. Thus in some embodiments the invention provides a method of determining the prognosis of a patient's cancer comprising determining the expression level of a panel of genes comprising at least 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 or more cell-cycle genes (e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 genes from Table 1), wherein high expression (or increased expression or overexpression) indicates an increased likelihood of recurrence or metastatic progression of the cancer. In some embodiments, the method comprises at least one of the following steps: (a) correlating high expression (or increased expression or overexpression) of the panel of genes to an increased likelihood of recurrence or metastatic progression; (b) concluding that the patient has an increased likelihood of recurrence or metastatic progression based at least in part on high expression (or increased expression or overexpression) of the panel of genes; or (c) communicating that the patient has an increased likelihood of recurrence or metastatic progression based at least in part on high expression (or increased expression or overexpression) of the panel of genes.

“Recurrence” and “metastatic progression” are terms well-known in the art and are used herein according to their known meanings. Because the methods of the invention can predict or determine a patient's likelihood of each, “recurrence,” “metastatic progression,” “cancer-specific death,” and “response to a particular treatment” are used interchangeably, unless specified otherwise, in the sense that a reference to one applies equally to the others. As an example, the meaning of “metastatic progression” may be cancer-type dependent, with metastatic progression in one form of renal cancer meaning something different from metastatic progression in another form of renal cancer. However, within each cancer-type and subtype “metastatic progression” is clearly understood to those skilled in the art. Because predicting recurrence and predicting metastatic progression are prognostic endeavors, “predicting prognosis” will often be used herein to refer to either or both. In these cases, a “poor prognosis” will generally refer to an increased likelihood of recurrence, metastatic progression, or both.

“Response” (e.g., response to a particular treatment regimen) is a well-known term in the art and is used herein according to its known meaning. As an example, the meaning of “response” may be cancer-type dependent, with response in some forms of renal cancer meaning something different from response in other forms of renal cancer. However, within each cancer-type and subtype “response” is clearly understood to those skilled in the art. For example, some objective criteria of response include Response Evaluation Criteria In Solid Tumors (RECIST), a set of published rules (e.g., changes in tumor size, etc.) that define when cancer patients improve (“respond”), stay the same (“stabilize”), or worsen (“progression”) during treatments. See, e.g., Eisenhauer et al., EUR. J. CANCER (2009) 45:228-247. “Response” can also include survival metrics (e.g., “disease-free survival” (DFS), “overall survival” (OS), etc). In some cases RECIST criteria can include: (a) Complete response (CR): disappearance of all metastases; (b) Partial response (PR): at least a 30% decrease in the sum of the largest diameter (LD) of the metastatic lesions, taking as reference the baseline sum LD; (c) Stable disease (SD): neither sufficient shrinkage to qualify for PR nor sufficient increase to qualify for PD taking as references the smallest sum LD since the treatment started; (d) Progressive disease (PD): at least a 20% increase in the sum of the LD of the target metastatic lesions taking as reference the smallest sum LD since the treatment started or the appearance of one or more new lesions.

As used herein, a patient has an “increased likelihood” of some clinical feature or outcome (e.g., recurrence or progression) if the probability of the patient having the feature or outcome exceeds some reference probability or value. The reference probability may be the probability of the feature or outcome across the general relevant patient population. For example, if the probability of recurrence in the general renal cancer population is X % and a particular patient has been determined by the methods of the present invention to have a probability of recurrence of Y %, and if Y>X, then the patient has an “increased likelihood” of recurrence. Alternatively, as discussed above, a threshold or reference value may be determined and a particular patient's probability of recurrence may be compared to that threshold or reference.

In some embodiments the method correlates the patient's specific expression or score (e.g., CCP score, combined score of CCP with clinical variables) to a specific probability (e.g., 10%, 15%, 20%, 25%, 30%, 35%, 40%, 45%, 50%, 55%, 60%, 65%, 70%, 75%, 80%, 85%, 90%, 95%, 99%, 100%) of the particular clinical event or outcome, e.g., recurrence, metastatic progression, or cancer-specific death (each optionally within a specific timeframe, e.g., 5 years, 10 years), or response to a particular treatment. In some embodiments the invention provides a method for determining a renal cancer patient's prognosis comprising: (1) determining from a patient sample the expression levels of a plurality of test genes, wherein the plurality of test genes comprises at least 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 or more cell-cycle genes (e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 genes from Table 1); (2) deriving a test value from the expression levels determined in (1), wherein the at least 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 or more cell-cycle genes (e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 genes from Table 1) contribute at least 25% to the test value; (3) comparing the test value to a reference value; and (4) assigning a likelihood of recurrence, metastatic progression, cancer-specific death, or response to a particular treatment based at least in part on the comparison in (3).

It has been determined that the choice of individual CCGs can often be less important than the overall CCP content/contribution of the prognostic gene expression panel. In other words, most CCGs have been found to be very good surrogates for each other. One way of assessing whether particular CCGs will serve well in the methods and compositions of the invention is by assessing their correlation with the mean expression of CCGs (e.g., all known CCGs, a specific set of CCGs, etc.). Those CCGs that correlate particularly well with the mean are expected to perform well in assays of the invention, e.g., because these will reduce noise in the assay.

In CCG signatures the particular CCGs assayed is often not as important as the total number of CCGs. The number of CCGs assayed can vary depending on many factors, e.g., technical constraints, cost considerations, the classification being made, the cancer being tested, the desired level of predictive power, etc. Increasing the number of CCGs assayed in a panel according to the invention is, as a general matter, advantageous because, e.g., a larger pool of mRNAs to be assayed means less “noise” caused by outliers and less chance of an assay error throwing off the overall predictive power of the test. However, cost and other considerations will generally limit this number and finding the optimal number of CCGs for a signature is desirable.

It has been discovered that the predictive power of a CCG signature often ceases to increase significantly beyond a certain number of CCGs. More specifically, the optimal number of CCGs in a signature (no) can be found wherever the following is true

(Pₙ₊₁−Pₙ)<C_{O},

wherein P is the predictive power (i.e., Pₙ is the predictive power of a signature with n genes and Pₙ₊₁ is the predictive power of a signature with n genes plus one) and Co is some optimization constant. Predictive power can be defined in many ways known to those skilled in the art including, but not limited to, the signature's p-value. Co can be chosen by the artisan based on his or her specific constraints. For example, if cost is not a critical factor and extremely high levels of sensitivity and specificity are desired, Co can be set very low such that only trivial increases in predictive power are disregarded. On the other hand, if cost is decisive and moderate levels of sensitivity and specificity are acceptable, Co can be set higher such that only significant increases in predictive power warrant increasing the number of genes in the signature.

Alternatively, a graph of predictive power as a function of gene number may be plotted and the second derivative of this plot taken. The point at which the second derivative decreases to some predetermined value (C_{O}′) may be the optimal number of genes in the signature.

It has been discovered that CCGs are particularly predictive in certain renal cancers. For example, a panel of 31 CCGs have been determined to be accurate in predicting metastatic progression in clear cell renal cell carcinoma (ccRCC) (EXAMPLE 1). Further, CCGs can potentially determine prognosis in other types of renal cancers, as summarized herein.

In some embodiments the panel comprises at least 3, 4, 5, 6, 7, 8, 9, 10, 15, 20, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 or more cell-cycle genes (e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 genes from Table 1). In some embodiments the panel comprises between 5 and 100 CCGs, between 7 and 40 CCGs, between 5 and 25 CCGs, between 10 and 20 CCGs, or between 10 and 15 CCGs. In some embodiments CCGs comprise at least a certain proportion of the panel. Thus in some embodiments the panel comprises at least 25%, 30%, 40%, 50%, 60%, 70%, 75%, 80%, 85%, 90%, 95%, 96%, 97%, 98%, or 99% CCGs. In some embodiments the CCGs are chosen from the group consisting of the genes listed in Tables 1. In some embodiments the panel comprises at least 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 or more cell-cycle genes (e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 genes from Table 1). In some embodiments the panel comprises all of the genes listed in Table 1.

It has further been discovered that CCG status synergistically adds to clinical parameters in prognosing cancer. In the case of ccRCC, for example, it has been discovered that a high level of gene expression of the genes in Table 1 is associated with an increased risk of ccRCC recurrence or metastatic progression in patients whose cancers show no evidence of lymph-vascular invasion, in patients with smaller tumors, and in younger patients. Because evaluating CCG expression levels can thus detect increased risk not detected using clinical parameters alone, the invention generally provides methods combining evaluating at least one clinical parameter with evaluating the status of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 or more cell-cycle genes (e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 genes from Table 1).

Often the clinical parameter is at least somewhat independently predictive of recurrence or metastatic progression and the addition of CCG status improves the predictive power. As used herein, “clinical parameter” and “clinical measure” refer to disease or patient characteristics that are typically applied to assess disease course and/or predict outcome. Examples of clinical parameters measured in renal cancer generally include tumor size, tumor stage, tumor grade, lymph node status and particularly evidence of lymph-vascular invasion, histology, performance status, type of surgery, histology of surgical margins, type of treatment, and age of onset. In renal cancer after surgical intervention, important clinical parameters include tumor size, evidence of lymph-vascular invasion, and Fuhrman nuclear grade.

Often certain clinical parameters are correlated with a particular disease character. For example, in cancer generally as well as in specific cancers, certain clinical parameters are correlated with, e.g., likelihood of recurrence or metastatic progression, prognosis for survival for a certain amount of time, likelihood of response to treatment generally or to a specific treatment, etc. In renal cancer some clinical parameters are such that their status (presence, absence, level, etc.) is associated with increased likelihood of recurrence or metastatic progression. Examples of such recurrence-associated parameters (some but not all of which are specific to renal cancer) include large tumor size, evidence of metastasis, advanced tumor stage, high Fuhrman nuclear grade, evidence of lymph-vascular invasion, and early age of onset. As used herein, “recurrence-associated clinical parameter” and “metastatic progression-associated clinical parameter” have their conventional meaning for each specific type and subtype of renal cancer, with which those skilled in the art are quite familiar. In fact, those skilled in the art are familiar with various recurrence-associated and metastatic progression-associated clinical parameters beyond those listed here.

Often a physician will assess more than one clinical parameter in a patient and make a more comprehensive evaluation for the disease characters of interest. Example 1 shows how CCG status can add to one particular grouping of clinical parameters used to determine risk of recurrence or metastatic progression in renal cancer.

In some embodiments clinical assessment is made before cytoreductive nephrectomy (e.g., using a biopsy sample) while in some embodiments it is made after (e.g., using the resected renal tumor sample). In some embodiments, a sample of one or more cells are obtained from a renal cancer patient before or after treatment for analysis according to the present invention. Renal cancer treatment currently applied in the art includes, e.g., radical nephrectomy, partial nephrectomy, regional lymphadenectomy, adrenalectomy, cryotherapy (cryoablation), radiofrequency ablation, arterial embolization, radiation therapy, targeted therapy with Sorafenib, Sunitinib, Temsirolimus, Everolimus, Bevacizumab, Pazopanib, or Axitinib, immunotherapy with cytokines including interleukin-2 (IL-2) and interferon-alpha, and some instances, conventional chemotherapy with vinblasine, floxuridine, 5-fluorouracil, cpecitabine, and gemcitabine. In some embodiments, one or more renal tumor cells from renal cancer tissue are obtained from a renal cancer patient during biopsy or nephrectomy and are used for analysis in the method of the present invention.

The results of any analyses according to the invention will often be communicated to physicians, genetic counselors and/or patients (or other interested parties such as researchers) in a transmittable form that can be communicated or transmitted to any of the above parties. Such a form can vary and can be tangible or intangible. The results can be embodied in descriptive statements, diagrams, photographs, charts, images or any other visual forms. For example, graphs showing expression or activity level or sequence variation information for various genes can be used in explaining the results. Diagrams showing such information for additional target gene(s) are also useful in indicating some testing results. The statements and visual forms can be recorded on a tangible medium such as papers, computer readable media such as floppy disks, compact disks, etc., or on an intangible medium, e.g., an electronic medium in the form of email or website on internet or intranet. In addition, results can also be recorded in a sound form and transmitted through any suitable medium, e.g., analog or digital cable lines, fiber optic cables, etc., via telephone, facsimile, wireless mobile phone, internet phone and the like.

Thus, the information and data on a test result can be produced anywhere in the world and transmitted to a different location. As an illustrative example, when an expression level, activity level, or sequencing (or genotyping) assay is conducted outside the United States, the information and data on a test result may be generated, cast in a transmittable form as described above, and then imported into the United States. Accordingly, the present invention also encompasses a method for producing a transmittable form of information on at least one of (a) expression level or (b) activity level for at least one patient sample. The method comprises the steps of (1) determining at least one of (a) or (b) above according to methods of the present invention; and (2) embodying the result of the determining step in a transmittable form. The transmittable form is the product of such a method.

Techniques for analyzing such expression, activity, and/or sequence data (indeed any data obtained according to the invention) will often be implemented using hardware, software or a combination thereof in one or more computer systems or other processing systems capable of effectuating such analysis.

Thus, the present invention further provides a system for determining gene expression in a tumor sample, comprising: (1) a sample analyzer for determining the expression levels of a panel of genes in a tumor sample including at least 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 or more cell-cycle genes (e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 genes from Table 1), wherein the sample analyzer contains the tumor sample which is from a patient identified as having renal cancer, or cDNA molecules from mRNA expressed from the panel of genes; (2) a first computer program for (a) receiving gene expression data on at least 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 or more test genes selected from the panel of genes, (b) weighting the determined expression of each of the test genes, and (c) combining the weighted expression to provide a test value, wherein at least 20%, 50%, at least 75% or at least 90% of the test genes are cell-cycle genes; and optionally (3) a second computer program for comparing the test value to one or more reference values each associated with a predetermined degree of risk of cancer recurrence or metastatic progression of the renal cancer. In some embodiments, the system further comprises a display module displaying the comparison between the test value to the one or more reference values, or displaying a result of the comparing step.

In a preferred embodiment, the amount of RNA transcribed from the panel of genes including test genes is measured in the tumor sample. In addition, the amount of RNA of one or more housekeeping genes in the tumor sample is also measured, and used to normalize or calibrate the expression of the test genes, as described above.

In some embodiments, the plurality of test genes includes at least 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 or more cell-cycle genes (e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 genes from Table 1), which constitute at least 50%, 75% or 80% of the plurality of test genes, and preferably 100% of the plurality of test genes.

The sample analyzer can be any instruments useful in determining gene expression, including, e.g., a sequencing machine, a real-time PCR machine, and a microarray instrument.

The computer-based analysis function can be implemented in any suitable language and/or browsers. For example, it may be implemented with C language and preferably using object-oriented high-level programming languages such as Visual Basic, SmallTalk, C++, and the like. The application can be written to suit environments such as the Microsoft Windows™ environment including Windows™ 98, Windows™ 2000, Windows™ NT, and the like. In addition, the application can also be written for the MacIntosh™, SUN™, UNIX or LINUX environment. In addition, the functional steps can also be implemented using a universal or platform-independent programming language. Examples of such multi-platform programming languages include, but are not limited to, hypertext markup language (HTML), JAVA™, JavaScript™, Flash programming language, common gateway interface/structured query language (CGI/SQL), practical extraction report language (PERL), AppleScript™ and other system script languages, programming language/structured query language (PL/SQL), and the like. Java™- or JavaScript™-enabled browsers such as HotJava™, Microsoft™ Explorer™, or Netscape™ can be used. When active content web pages are used, they may include Java™ applets or ActiveX™ controls or other active content technologies.

The analysis function can also be embodied in computer program products and used in the systems described above or other computer- or internet-based systems. Accordingly, another aspect of the present invention relates to a computer program product comprising a computer-usable medium having computer-readable program codes or instructions embodied thereon for enabling a processor to carry out gene status analysis. These computer program instructions may be loaded onto a computer or other programmable apparatus to produce a machine, such that the instructions which execute on the computer or other programmable apparatus create means for implementing the functions or steps described above. These computer program instructions may also be stored in a computer-readable memory or medium that can direct a computer or other programmable apparatus to function in a particular manner, such that the instructions stored in the computer-readable memory or medium produce an article of manufacture including instructions which implement the analysis. The computer program instructions may also be loaded onto a computer or other programmable apparatus to cause a series of operational steps to be performed on the computer or other programmable apparatus to produce a computer implemented process such that the instructions which execute on the computer or other programmable apparatus provide steps for implementing the functions or steps described above.

Thus one aspect of the present invention provides a system for determining whether a patient has increased likelihood of recurrence or metastatic progression. Generally speaking, the system comprises (1) one or more computer programs for receiving, storing, and/or retrieving a patient's gene status data (e.g., expression level, activity level, variants) and optionally clinical parameter data (e.g., tumor size, Fuhrman nuclear grade, lymph-vascular invasion, age of onset, etc.); (2) one or more computer programs for querying this patient data; (3) one or more computer programs for concluding whether there is an increased likelihood of recurrence or metastatic progression based on this patient data; and optionally (4) one or more computer programs for outputting/displaying this conclusion. In some embodiments this computer program for outputting the conclusion may comprise a computer program for informing a health care professional of the conclusion.

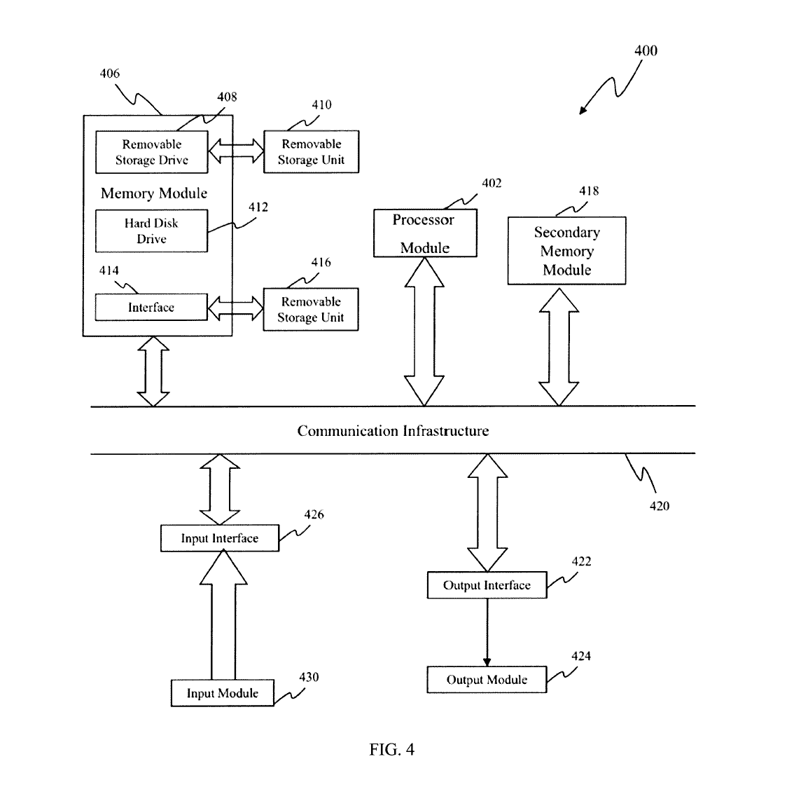

One example of such a computer system is the computer system [400] illustrated in FIG. 4. Computer system [400] may include at least one input module [430] for entering patient data into the computer system [400]. The computer system [400] may include at least one output module [424] for indicating whether a patient has an increased or decreased likelihood of response and/or indicating suggested treatments determined by the computer system [400]. Computer system [400] may include at least one memory module [406] in communication with the at least one input module [430] and the at least one output module [424].

The at least one memory module [406] may include, e.g., a removable storage drive [408], which can be in various forms, including but not limited to, a magnetic tape drive, a floppy disk drive, a VCD drive, a DVD drive, an optical disk drive, etc. The removable storage drive [408] may be compatible with a removable storage unit [410] such that it can read from and/or write to the removable storage unit [410]. Removable storage unit [410] may include a computer usable storage medium having stored therein computer-readable program codes or instructions and/or computer readable data. For example, removable storage unit [410] may store patient data. Example of removable storage unit [410] are well known in the art, including, but not limited to, Universal Serial Bus solid state memory drives (i.e., “USB thumb drives”), floppy disks, magnetic tapes, optical disks, and the like. The at least one memory module [406] may also include a hard disk drive [412], which can be used to store computer readable program codes or instructions, and/or computer readable data.

In addition, as shown in FIG. 4, the at least one memory module [406] may further include an interface [414] and a removable storage unit [416] that is compatible with interface [414] such that software, computer readable codes or instructions can be transferred from the removable storage unit [416] into computer system [400]. Examples of interface [414] and removable storage unit [416] pairs include, e.g., removable memory chips (e.g., EPROMs or PROMs) and sockets associated therewith, program cartridges and cartridge interface, and the like. Computer system [400] may also include a secondary memory module [418], such as random access memory (RAM).

Computer system [400] may include at least one processor module [402]. It should be understood that the at least one processor module [402] may consist of any number of devices. The at least one processor module [402] may include a data processing device, such as a microprocessor or microcontroller or a central processing unit. The at least one processor module [402] may include another logic device such as a DMA (Direct Memory Access) processor, an integrated communication processor device, a custom VLSI (Very Large Scale Integration) device or an ASIC (Application Specific Integrated Circuit) device. In addition, the at least one processor module [402] may include any other type of analog or digital circuitry that is designed to perform the processing functions described herein.

As shown in FIG. 4, in computer system [400], the at least one memory module [406], the at least one processor module [402], and secondary memory module [418] are all operably linked together through communication infrastructure [420], which may be a communications bus, system board, cross-bar, etc.). Through the communication infrastructure [420], computer program codes or instructions or computer readable data can be transferred and exchanged. Input interface [426] may operably connect the at least one input module [426] to the communication infrastructure [420]. Likewise, output interface [422] may operably connect the at least one output module [424] to the communication infrastructure [420].

The at least one input module [430] may include, for example, a keyboard, mouse, touch screen, scanner, and other input devices known in the art. The at least one output module [424] may include, for example, a display screen, such as a computer monitor, TV monitor, or the touch screen of the at least one input module [430]; a printer; and audio speakers. Computer system [400] may also include, modems, communication ports, network cards such as Ethernet cards, and newly developed devices for accessing intranets or the internet.

The at least one memory module [406] may be configured for storing patient data entered via the at least one input module [430] and processed via the at least one processor module [402]. Patient data relevant to the present invention may include expression level, activity level, copy number and/or sequence information for a test gene or genes. Patient data relevant to the present invention may also include clinical parameters relevant to the patient's disease. Any other patient data a physician might find useful in making treatment decisions/recommendations may also be entered into the system, including but not limited to age, gender, and race/ethnicity and lifestyle data such as diet information. Other possible types of patient data include symptoms currently or previously experienced, patient's history of illnesses, medications, and medical procedures.

The at least one memory module [406] may include a computer-implemented method stored therein. The at least one processor module [402] may be used to execute software or computer-readable instruction codes of the computer-implemented method. The computer-implemented method may be configured to, based upon the patient data, indicate whether the patient has an increased likelihood of recurrence, metastatic progression or response to any particular treatment, generate a list of possible treatments, etc.

In certain embodiments, the computer-implemented method may be configured to identify a patient as having or not having an increased likelihood of recurrence or metastatic progression. For example, the computer-implemented method may be configured to inform a physician that a particular patient has an increased likelihood of recurrence or metastatic progression. Alternatively or additionally, the computer-implemented method may be configured to actually suggest a particular course of treatment based on the answers to/results for various queries.

FIG. 5 illustrates one embodiment of a computer-implemented method [500] of the invention that may be implemented with the computer system [400] of the invention. The method [500] begins with one of two queries ([510]& [511]), either sequentially or substantially simultaneously. If the answer to/result for any of these queries is “Yes” [520], the method concludes [530] that the patient has an increased likelihood of recurrence or metastatic progression. If the answer to/result for all of these queries is “No” [521], the method concludes [531] that the patient does not have an increased likelihood of recurrence or metastatic progression. The method [500] may then proceed with more queries, make a particular treatment recommendation ([540], [541]), or simply end.

When the queries are performed sequentially, they may be made in the order suggested by FIG. 5 or in any other order. Whether subsequent queries are made can also be dependent on the results/answers for preceding queries. In some embodiments of the method illustrated in FIG. 5, for example, the method asks about clinical parameters [511] first and, if the patient has one or more clinical parameters identifying the patient as at increased risk for recurrence or metastatic progression then the method concludes such [530] or optionally confirms by querying CCG status, while if the patient has no such clinical parameters then the method proceeds to ask about CCG status [510]. As mentioned above, the preceding order of queries may be modified. In some embodiments an answer of “yes” to one query (e.g., [511]) prompts one or more of the remaining queries to confirm that the patient has increased risk of recurrence or metastatic progression.

In some embodiments, the computer-implemented method of the invention [500] is open-ended. In other words, the apparent first step [510 or 511] in FIG. 5 may actually form part of a larger process and, within this larger process, need not be the first step/query. Additional steps may also be added onto the core methods discussed above. These additional steps include, but are not limited to, informing a health care professional (or the patient itself) of the conclusion reached; combining the conclusion reached by the illustrated method [500] with other facts or conclusions to reach some additional or refined conclusion regarding the patient's diagnosis, prognosis, treatment, etc.; making a recommendation for treatment (e.g., “patient should/should not undergo cytokine immunotherapy”); additional queries about additional biomarkers, clinical parameters, or other useful patient information (e.g., age at diagnosis, general patient health, etc.).

Regarding the above computer-implemented method [500], the answers to the queries may be determined by the method instituting a search of patient data for the answer. For example, to answer the respective queries [510 & 511], patient data may be searched for CCG status (e.g., CCG expression level data), or clinical parameters (e.g., tumor size, Fuhrman nuclear score, evidence of lymph-vascular invasion, etc.). If such a comparison has not already been performed, the method may compare these data to some reference in order to determine if the patient has an abnormal (e.g., elevated, low, negative) status. Additionally or alternatively, the method may present one or more of the queries [510 & 511] to a user (e.g., a physician) of the computer system [400]. For example, the questions [510 & 511] may be presented via an output module [424]. The user may then answer “Yes” or “No” via an input module [430]. The method may then proceed based upon the answer received. Likewise, the conclusions [530, 531] may be presented to a user of the computer-implemented method via an output module [424].

Thus in some embodiments the invention provides a method comprising: accessing information on a patient's CCG status, and/or clinical parameters stored in a computer-readable medium; querying this information to determine at least one of whether a sample obtained from the patient shows increased expression of at least one CCG, and/or whether the patient has a recurrence-associated clinical parameter; outputting [or displaying] the sample's CCG expression status, and/or the patient's recurrence-associated clinical parameter status. As used herein in the context of computer-implemented embodiments of the invention, “displaying” means communicating any information by any sensory manner. Examples include, but are not limited to, visual displays, e.g., on a computer screen or on a sheet of paper printed at the command of the computer, and auditory displays, e.g., computer generated or recorded auditory expression of a patient's genotype.

As discussed at length above, recurrence-associated, or metastatic cancer progression-associated clinical parameters combined with elevated CCG status indicate a significantly increased likelihood of recurrence. Thus some embodiments provide a computer-implemented method of determining whether a patient has an increased likelihood of recurrence comprising accessing information on a patient's clinical parameters and CCG status (e.g., from a tumor sample obtained from the patient) stored in a computer-readable medium; querying this information to determine at least one of whether the patient has a recurrence-associated, or metastatic cancer progression-associated clinical parameter; querying this information to determine whether a sample obtained from the patient shows increased expression of at least one CCG; outputting (or displaying) an indication that the patient has an increased likelihood of recurrence or metastatic progression if the patient has a low/negative recurrence-associated, or metastatic cancer progression-associated clinical parameter and the sample shows increased expression of at least one CCG. Some embodiments further comprise displaying clinical parameters (or their values) and/or the CCGs and their status (including, e.g., expression levels), optionally together with an indication of whether the CCG status and/or clinical parameter indicates increased likelihood of risk.

The practice of the present invention may also employ conventional biology methods, software and systems. Computer software products of the invention typically include computer readable media having computer-executable instructions for performing the logic steps of the method of the invention. Suitable computer readable medium include floppy disk, CD-ROM/DVD/DVD-ROM, hard-disk drive, flash memory, ROM/RAM, magnetic tapes and etc. Basic computational biology methods are described in, for example, Setubal et al., INTRODUCTION TO COMPUTATIONAL BIOLOGY METHODS (PWS Publishing Company, Boston, 1997); Salzberg et al. (Ed.), COMPUTATIONAL METHODS IN MOLECULAR BIOLOGY, (Elsevier, Amsterdam, 1998); Rashidi & Buehler, BIOINFORMATICS BASICS: APPLICATION IN BIOLOGICAL SCIENCE AND MEDICINE (CRC Press, London, 2000); and Ouelette & Bzevanis, BIOINFORMATICS: A PRACTICAL GUIDE FOR ANALYSIS OF GENE AND PROTEINS (Wiley & Sons, Inc., 2^{rd} ed., 2001); see also, U.S. Pat. No. 6,420,108.

The present invention may also make use of various computer program products and software for a variety of purposes, such as probe design, management of data, analysis, and instrument operation. See U.S. Pat. Nos. 5,593,839; 5,795,716; 5,733,729; 5,974,164; 6,066,454; 6,090,555; 6,185,561; 6,188,783; 6,223,127; 6,229,911 and 6,308,170. Additionally, the present invention may have embodiments that include methods for providing genetic information over networks such as the Internet as shown in U.S. Ser. No. 10/197,621 (U.S. Pub. No. 20030097222); Ser. No. 10/063,559 (U.S. Pub. No. 20020183936), Ser. No. 10/065,856 (U.S. Pub. No. 20030100995); Ser. No. 10/065,868 (U.S. Pub. No. 20030120432); Ser. No. 10/423,403 (U.S. Pub. No. 20040049354).

Techniques for analyzing such expression, activity, and/or sequence data (indeed any data obtained according to the invention) will often be implemented using hardware, software or a combination thereof in one or more computer systems or other processing systems capable of effectuating such analysis.

Thus one aspect of the present invention provides systems related to the above methods of the invention. In one embodiment the invention provides a system for determining gene expression in a tumor sample, comprising:
(1) a sample analyzer for determining the expression levels in a sample of a panel of genes including at least 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 or more cell-cycle genes (e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 genes from Table 1), wherein the sample analyzer contains the sample, RNA from the sample and expressed from the panel of genes, or DNA synthesized from said RNA;
(2) a first computer program for
   (a) receiving gene expression data on at least 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 or more test genes selected from the panel of genes,
   (b) weighting the determined expression of each of the test genes with a predefined coefficient, and
   (c) combining the weighted expression to provide a test value, wherein the combined weight given to said at least 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 or more cell-cycle genes (e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 genes from Table 1) is at least 40% (or 50%, 60%, 70%, 80%, 90%, 95% or 100%) of the total weight given to the expression of all of said plurality of test genes; and optionally
      In some embodiments at least 20%, 50%, 75%, or 90% of said plurality of test genes are CCGs. In some embodiments the sample analyzer contains reagents for determining the expression levels in the sample of said panel of genes including at least 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 or more cell-cycle genes (e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 genes from Table 1). In some embodiments the sample analyzer contains CCG-specific reagents as described below.

In another embodiment the invention provides a system for determining gene expression in a tumor sample, comprising: (1) a sample analyzer for determining the expression levels of a panel of genes in a tumor sample including at least 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 or more cell-cycle genes (e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 genes from Table 1), wherein the sample analyzer contains the tumor sample which is from a patient identified as having renal cancer, RNA from the sample and expressed from the panel of genes, or DNA synthesized from said RNA; (2) a first computer program for (a) receiving gene expression data on at least 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 or more test genes selected from the panel of genes, (b) weighting the determined expression of each of the test genes with a predefined coefficient, and (c) combining the weighted expression to provide a test value, wherein the combined weight given to said at least 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 or more cell-cycle genes (e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 genes from Table 1) is at least 40% (or 50%, 60%, 70%, 80%, 90%, 95% or 100%) of the total weight given to the expression of all of said plurality of test genes; and optionally (3) a second computer program for comparing the test value to one or more reference values each associated with a predetermined degree of risk of cancer recurrence or metastatic progression of the renal cancer. In some embodiments at least 20%, 50%, 75%, or 90% of said plurality of test genes are CCGs. In some embodiments the system comprises a computer program for determining the patient's prognosis and/or determining (including quantifying) the patient's degree of risk of cancer recurrence or metastatic progression based at least in part on the comparison of the test value with said one or more reference values.

In some embodiments, the system further comprises a display module displaying the comparison between the test value and the one or more reference values, or displaying a result of the comparing step, or displaying the patient's prognosis and/or degree of risk of cancer recurrence or metastatic progression.

In a preferred embodiment, the amount of RNA transcribed from the panel of genes including test genes (and/or DNA reverse transcribed therefrom) is measured in the sample. In addition, the amount of RNA of one or more housekeeping genes in the sample (and/or DNA reverse transcribed therefrom) is also measured, and used to normalize or calibrate the expression of the test genes, as described above.

In some embodiments, the plurality of test genes includes at least 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 or more cell-cycle genes (e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 genes from Table 1), which constitute at least 50%, 75% or 80% of the plurality of test genes, and preferably 100% of the plurality of test genes. Thus in some embodiments the plurality of test genes comprises at least some number of CCGs (e.g., at least 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 genes from Table 1) and this plurality of CCGs comprises the top 2, 3, 4, 5, 6, 7, 8, 9, 10, 11, 12, 13, 14, 15, 16, 17, 18, 19, 20, 21, 22, 23, 24, 25, 26, 27, 28, 29, or 30 genes from Table 1.

The sample analyzer can be any instrument useful in determining gene expression, including, e.g., a sequencing machine (e.g., Illumina HiSeq™, Ion Torrent PGM, ABI SOLiD™ sequencer, PacBio RS, Helicos Heliscope™, etc.), a real-time PCR machine (e.g., ABI 7900, Fluidigm BioMark™, etc.), a microarray instrument, etc.

In one aspect, the present invention provides methods of treating a cancer patient comprising obtaining CCG status information (e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 genes from Table 1), and recommending, prescribing or administering a treatment for the cancer patient based on the CCG status. In some embodiments, the method further includes obtaining clinical parameter information, from the patient and treating the patient with a particular treatment based on the CCG status, and/or clinical parameter. For example, the invention provides a method of treating a cancer patient comprising:
(1) determining the status of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 or more cell-cycle genes (e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 genes from Table 1);
(2) determining the status of at least on clinical parameter; and
(3) recommending, prescribing or administering either
   (a) an active (including aggressive) treatment if the patient has increased expression of the CCGs, and/or a recurrence-associated, or metastatic progression-associated clinical parameter, or
   (b) a passive (or less aggressive) treatment if the patient does not have increased expression of the CCGs, and/or does not exhibit a recurrence-associated, or metastatic progression-associated clinical parameter.
      In some embodiments, the determining steps comprise receiving a report communicating the relevant status (e.g., CCG status). In some embodiments this report communicates such status in a qualitative manner (e.g., “high” or “increased” expression). In some embodiments this report communicates such status indirectly by communicating a score (e.g., prognosis score, recurrence score, metastatic progression score, or combined score as discussed above, etc.) that incorporates such status.

Whether a treatment is aggressive or not will generally depend on the cancer-type, the age of the patient, etc. For example, in renal cancer adjuvant targeted therapy is a common aggressive treatment given to complement the less aggressive standards of surgery and immunotherapy therapy. Those skilled in the art are familiar with various other aggressive and less aggressive treatments for each type of cancer. “Active treatment” in renal cancer is well-understood by those skilled in the art and, as used herein, has the conventional meaning in the art. Generally speaking, active treatment in renal cancer is anything other than “watchful waiting.” Active treatments currently applied in the art of renal cancer therapy include, e.g., radical nephrectomy, partial nephrectomy, regional lymphadenectomy, adrenalectomy, cryotherapy (cryoablation), radiofrequency ablation, arterial embolization, radiation therapy, targeted therapy with antiangiogenic agents, and/or treatment with mTOR kinase inhibitors, and particularly drugs such as Sorafenib, Sunitinib, Temsirolimus, Everolimus, Bevacizumab, Pazopanib, or Axitinib, immunotherapy with cytokines including interleukin-2 (IL-2) and interferon-alpha, and in some instances, conventional chemotherapy with vinblasine, floxuridine, 5-fluorouracil, cpecitabine, and gemcitabine, etc. Each treatment option carries with it certain risks as well as side-effects of varying severity. Thus, it is common for doctors, depending on the age and general health of the patient diagnosed with renal cancer, to recommend a regime of “watchful-waiting,” particularly after the patient has undergone cytoreductive nephrectomy.

“Watchful-waiting,” also called “active surveillance,” also has its conventional meaning in the art. This generally means observation and regular monitoring without invasive treatment. Watchful-waiting is sometimes used, e.g., when an early stage, slow-growing renal cancer is found in an older patient. Watchful-waiting may also be suggested when the risks of initial surgery or follow-on surgeries, and adjuvant therapies, including immunotherapy, targeted therapy, or conventional chemotherapy, outweigh the possible benefits. Other treatments can be started if symptoms develop, or if there are signs that the cancer growth is accelerating (e.g., metastatic tumors rapidly increasing in size, etc.).

Although patients who choose watchful-waiting avoid the risks of surgery or various adjuvant therapies, watchful-waiting carries its own risks, e.g., increased risk of metastasis and metastatic progression. For younger patients, a trial of active surveillance may not mean avoiding treatment altogether, but may reasonably allow a delay of a few years or more, during which time the quality of life impact of active treatment can be avoided. Published data to date suggest that carefully selected patients will not miss a window for cure with this approach with some slow growing cancers. Additional health problems that develop with advancing age during the observation period can also make it harder to undergo surgery and more aggressive adjuvant therapy. Thus it is clinically important to carefully determine which renal cancer patients are good candidates for watchful-waiting and which patients should receive active treatment.

Thus, the invention provides a method of treating a renal cancer patient or providing guidance to the treatment of a patient. In this method, the status of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 or more cell-cycle genes (e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 genes from Table 1), and/or at least one recurrence-associated or metastatic progression-associated clinical parameter is determined, and (a) active treatment is recommended, initiated or continued if a sample from the patient has an elevated status for the CCGs or the patient has at least one recurrence-associated or metastatic progression-associated clinical parameter, or (b) watchful-waiting is recommended, initiated, or continued if the patient has neither an elevated status for the CCGs, nor a recurrence-associated or metastatic progression-associated clinical parameter. In certain embodiments the CCG status and clinical parameter(s) may indicate not just that active treatment is recommended, but that a particular active treatment is preferable for the patient (including relatively aggressive treatments such as, e.g., radical nephrectomy or aggressive adjuvant therapy).

In general, conventional chemotherapy, radiotherapy, hormonal therapy, etc. after nephrectomy is not the standard of care in renal cancer. More often cytokine immunotherapies or targeted therapies are prescribed. According to the present invention, however, physicians may be able to determine which renal cancer patients have particularly aggressive disease and thus should receive more aggressive forms of adjuvant therapy. Thus in one embodiment, the invention provides a method of treating a patient (e.g., a renal cancer patient) comprising determining the status of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 or more cell-cycle genes (e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 genes from Table 1) and the status of at least one recurrence-associated or metastatic progression-associated clinical parameter, and initiating a particular type of adjuvant therapy after nephrectomy if a sample from the patient has an elevated status for the CCGs and/or the patient has at least one recurrence-associated or metastatic progression-associated clinical parameters.

In one aspect, the invention provides compositions for use in the above methods. Such compositions include, but are not limited to, nucleic acid probes hybridizing to a set of CCGs (or to any nucleic acids encoded thereby or complementary thereto); nucleic acid primers and primer pairs suitable for amplifying all or a portion of a set of CCGs or any nucleic acids encoded thereby; antibodies binding immunologically to a polypeptide encoded by a set of CCGs; probe sets comprising a plurality of said nucleic acid probes, nucleic acid primers, antibodies, and/or polypeptides; microarrays comprising any of these; kits comprising any of these; etc. In some aspects, the invention provides computer methods, systems, software and/or modules for use in the above methods.

In some embodiments the invention provides a set of probes comprising isolated oligonucleotides capable of selectively hybridizing to at least 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 genes from Table 1. The terms “probe” and “oligonucleotide” (also “oligo”), when used in the context of nucleic acids, interchangeably refer to a relatively short nucleic acid fragment or sequence. The invention also provides primers useful in the methods of the invention. “Primers” are probes capable, under the right conditions and with the right companion reagents, of selectively amplifying a target nucleic acid (e.g., a target gene). In the context of nucleic acids, “probe” is used herein to encompass “primer” since primers can generally also serve as probes.

The probe can generally be of any suitable size/length. In some embodiments the probe has a length from about 8 to 200, 15 to 150, 15 to 100, 15 to 75, 15 to 60, or 20 to 55 bases in length. They can be labeled with detectable markers with any suitable detection marker including but not limited to, radioactive isotopes, fluorophores, biotin, enzymes (e.g., alkaline phosphatase), enzyme substrates, ligands and antibodies, etc. See Jablonski et al., NUCLEIC ACIDS RES. (1986) 14:6115-6128; Nguyen et al., BIOTECHNIQUES (1992) 13:116-123; Rigby et al., J. MOL. BIOL. (1977) 113:237-251. Indeed, probes may be modified in any conventional manner for various molecular biological applications. Techniques for producing and using such oligonucleotide probes are conventional in the art.

Probes according to the invention can be used in the hybridization/amplification/detection techniques discussed above. Thus, some embodiments of the invention comprise probe sets suitable for use in a microarray in detecting, amplifying and/or quantitating a plurality of CCGs. In some embodiments the probe sets have a certain proportion of their probes directed to CCGs—e.g., a probe set consisting of 10%, 20%, 30%, 40%, 50%, 55%, 60%, 65%, 70%, 75%, 80%, 85%, 90%, 95%, 96%, 97%, 98%, 99%, or 100% probes specific for CCGs. In some embodiments the probe set comprises probes directed to at least 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 genes from Table 1. Such probe sets can be incorporated into high-density arrays comprising 5,000, 10,000, 20,000, 50,000, 100,000, 200,000, 300,000, 400,000, 500,000, 600,000, 700,000, 800,000, 900,000, or 1,000,000 or more different probes. In other embodiments the probe sets comprise primers (e.g., primer pairs) for amplifying nucleic acids comprising at least 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 genes from Table 1.

In another aspect of the present invention, a kit is provided for practicing the prognosis of the present invention. The kit may include a carrier for the various components of the kit. The carrier can be a container or support, in the form of, e.g., bag, box, tube, rack, and is optionally compartmentalized. The carrier may define an enclosed confinement for safety purposes during shipment and storage. The kit includes various components useful in determining the status of 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 or more cell-cycle genes (e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 genes from Table 1) and one or more housekeeping gene markers, using the above-discussed detection techniques. For example, the kit many include oligonucleotides specifically hybridizing under high stringency to mRNA or cDNA of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 genes from Table 1. Such oligonucleotides can be used as PCR primers in RT-PCR reactions, or hybridization probes. In some embodiments the kit comprises reagents (e.g., probes, primers, and or antibodies) for determining the expression level of a panel of genes, where said panel comprises at least 25%, 30%, 40%, 50%, 60%, 75%, 80%, 90%, 95%, 99%, or 100% CCGs (e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 genes from Table 1). In some embodiments the kit consists of reagents (e.g., probes, primers, and or antibodies) for determining the expression level of no more than 2500 genes, wherein at least 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 or more of these genes are cell-cycle genes (e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 genes from Table 1).

The oligonucleotides in the detection kit can be labeled with any suitable detection marker including but not limited to, radioactive isotopes, fluorephores, biotin, enzymes (e.g., alkaline phosphatase), enzyme substrates, ligands and antibodies, etc. See Jablonski et al., Nucleic Acids Res., 14:6115-6128 (1986); Nguyen et al., Biotechniques, 13:116-123 (1992); Rigby et al., J. Mol. Biol., 113:237-251 (1977). Alternatively, the oligonucleotides included in the kit are not labeled, and instead, one or more markers are provided in the kit so that users may label the oligonucleotides at the time of use.

In another embodiment of the invention, the detection kit contains one or more antibodies selectively immunoreactive with one or more proteins encoded by 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 or more cell-cycle genes (e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 genes from Table 1) or optionally any additional markers. Examples include antibodies that bind immunologically to a protein encoded by a gene in Table 1. Methods for producing and using such antibodies have been described above in detail.

Various other components useful in the detection techniques may also be included in the detection kit of this invention. Examples of such components include, but are not limited to, Taq polymerase, deoxyribonucleotides, dideoxyribonucleotides, other primers suitable for the amplification of a target DNA sequence, RNase A, and the like. In addition, the detection kit preferably includes instructions on using the kit for practice the prognosis method of the present invention using human samples.

### Example 1

The following cell cycle gene (CCG) signature (i.e., the expression status of the 31 CCGs in Table 1) was tested for predicting metastatic progression of ccRCC after cytoreductive nephrectomy.

### Introduction:

CCP expression scores have the potential to predict adverse outcomes in a variety of cancers. This study was designed to test whether CCP expression scores have prognostic value for renal cancers. In particular, the study described below was designed to determine whether the CCP expression score can predict metastatic cancer progression of ccRCC following cytoreductive nephrectomy.

### Study Design:

This is a case-control study of CCP scores in ccRCC patients who had cytoreductive nephrectomy. Cases developed metastatic progression within 5 years of surgery, while controls had no evidence of disease recurrence within 5 years. From 68 patients, 64 were eligible (26 cases and 38 controls). At least 4.5 years of clinical follow up were required. One case with positive margins and three patients having clear cell type pathology with partial sarcomatoid differentiation were excluded from the analysis. Patients with local recurrence only were accepted as controls. No restriction was placed on Fuhrman Nuclear grade (FNG I-IV). No patients had neo-adjuvant or adjuvant treatment. In this study, we used logistic regression to test the association between CCP score and metastatic cancer progression, while adjusting for clinical covariates.

### Analysis:

CCP Scores:

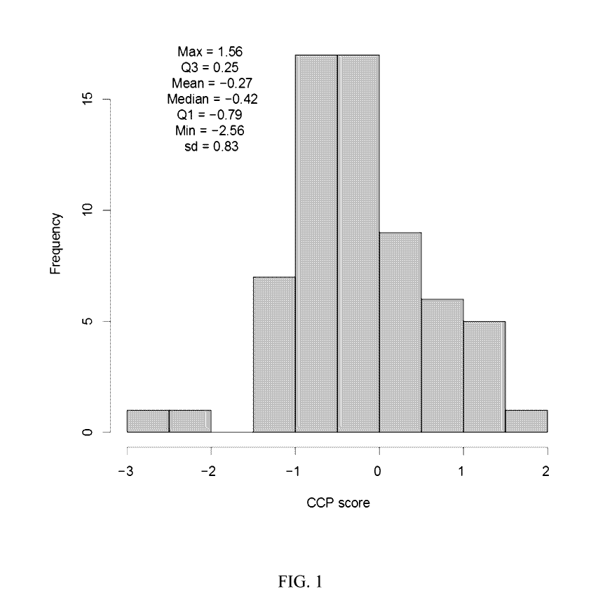

68 patient samples were received and analyzed on ProsAssay4 by the Prolaris process in the Myriad Research laboratory, and all produced good quality CCP scores. In order to calculate CCP scores, new priors were calculated for kidney tissue using the 68 samples in this cohort. FIG. 1 provides the distribution of CCP scores among eligible patients. The distribution of CCP scores was approximately normal (Shapiro-Wilk test under null hypothesis of normality, p-value 0.039) and did not display the right-sided skew observed in prostate cohorts (FIG. 1).

Eligible Sample Set:

Of the 68 samples, 4 were excluded for the following reasons:
One patient was excluded because it was a case with positive margins
Three samples (one case and two controls) were excluded because they showed clear cell type pathology with partial sarcomatoid differentiation. Also, the two highest CCP scores: 2.41 and 2.45, were obtained for patients with cells showing 20% and 90% sarcomatoid differentiation respectively. According to Dr. Sangale, this is not a surprise, cells of that type are from patients with the most aggressive tumors.

After the 4.5 years of clinical follow-up required, some controls were followed for up to 9.34 more years. Three controls had metastasis at 8.44, 8.61 and 9.28 years of follow up.

Modeling and Clinical Data:

Sex, age at surgery, TNM stage, follow up since surgery/time to metastasis, tumor size, nuclear grade, lymph-vascular invasion and smoking status were available for each patient, and were coded as follows:
Sex: Sex was coded as a binary variable (M=male, F=female);
Age at surgery: Age at surgery was reported in years and used as a quantitative variable;
Pathological stage: The categories reported for T-stage were: T2a, T2b, T3a and T3b. Also, all the patients were known to be diagnosed with lymph node negative and non metastasized cancer. Since the TNM system created by the America Joint Committee Cancer (AJCC) is the most commonly used staging system for kidney cancer (American Cancer Society, Kidney cancer (Adult)—Renal cell carcinoma. Atlanta, Ga. American Cancer Society, 2012), it was decided to group patients by TNM stage. Stage II for T2,N0,M0 and stage III for T3, N0, M0;
Time to metastasis (cases)/Last follow up since surgery (controls): The number of years from surgery to metastatic progression for cases and time from surgery until last follow up for controls;
Tumor size: Tumor size in cm was coded as a quantitative variable;
Nuclear Grade: The categories reported for Fuhrman nuclear grade were 1, 2, 3 and 4. It was decided to code Nuclear grade as a binary variable following Lang et al. (Lang et al., Multicenter determination of optimal interobserver agreement using the Fuhrman grading system for renal cell carcinoma; Atlanta, Ga., American Cancer Society. 2004) who claim that collapsing of the Fuhrman grade system into a low-grade group (Grade 1-2) and a high-grade group (Grade 3-4) improves interobserver agreement while preserving the independent prognostic value of nuclear grade;
Lymph-vascular invasion: Lymph-vascular invasion was used as a binary variable (No=cancer has not spread to the blood vessels and/or lymphatics, Yes=cancer has spread to the blood vessels and/or lymphatics); and
Smoking status: Smoking status was coded as a binary variable (Y=Yes, N=No).

Statistical Methods:

This study evaluated the association of CCP and clinical parameters with metastatic progression of the cancer modeled as the response variable. The prognostic value of CCP and clinical variables was evaluated in terms of p-values and odd-ratios from univariate and multivariate logistic regression models. The test statistic was the change in the likelihood deviance metric between the full model and the appropriate reduced model. Odds ratios were calculated to measure the risk of metastatic cancer for a one-unit increase in the corresponding variable. Statistical analyses were conducted using the R software environment (version 2.14.1, December 2011, R Development Core Team) and SAS 9.2 (SAS Institute, Cary N.C.). P-values were considered significant at a two-sided significance level of 0.05.

### Results:

Summary Measures:

TABLE 3
Clinical characteristics of patients eligible for analysis.

| | | Cases | Controls |
|---|---|---|---|
| Sex | Female | 7 | 16 (70%) |
| | Male | 22 | 19 (46%) |
| Age at surgery (years) | Q1 | 55.85 | 50.22 |
| | Median | 61.03 | 59.31 |
| | Mean | 61.35 | 57.92 |
| | Q3 | 69.58 | 64.96 |
| TNM stage | II | 6 | 17 (74%) |
| | III | 20 | 21 (51%) |
| Time to metastasis | Q1 | 1.06 | |
| | Median | 1.68 | |
| | Mean | 2.65 | |
| | Q3 | 3.69 | |
| Last follow up since | Q1 | | 5.88 |
| surgery | Median | | 6.69 |
| | Mean | | 7.76 |
| | Q3 | | 9.28 |
| Surgery | Partial | 0 (0%) | 2 (100%) |
| | Radical | 26 | 36 (58%) |
| Surgical Margins | Positive | 0 (0%) | 2 (100%) |
| | Uninvolved | 26 | 36 (58%) |
| Tumor size (cm) | Q1 | 7.5 | 6.4 |
| | Median | 9.75 | 8 |
| | Mean | 9.48 | 7.84 |
| | Q3 | 11 | 9.5 |
| Nuclear grade | Low-grade | 7 | 23 (77%) |
| | High-grade | 19 | 15 (44%) |
| Lymph-vascular | YES | 17 | 11 (39%) |
| invasion | NO | 9 | 27 (75%) |
| Smoking status | YES | 14 | 21 (60%) |
| | NO | 12 | 17 (59%) |
| CCP score | Q1 | −0.49 | −0.84 |
| | Median | −0.11 | −0.52 |
| | Mean | 0.075 | −0.5 |
| | Q3 | 1.56 | 1.22 |

Univariate Analysis:

In univariate analysis, the following variables reached significance at 5% level: lymph-vascular invasion (OR=4.64, p-value=0.0050), CCP (OR=2.65, p-value=0.0091) and nuclear grade (OR=4.16, p-value=0.0099). The summary of univariate analyses conducted is in Table 4.

TABLE 4
Univariate logistic regression models for some clinical variables

| Variable | Odds ratio | 95% CI for OR | P-value | AUC |
|---|---|---|---|---|
| Lymph-vascular invasion | 4.64 | (1.63, 5.89) | 0.0050 | 0.68 |
| (Yes versus No) | | | | |
| CCP | 2.65 | (1.34, 5.89) | 0.0091 | 0.68 |
| Nuclear grade (high | 4.16 | (1.46, 12.97) | 0.0099 | 0.67 |
| versus low) | | | | |
| Tumor size | 1.19 | (1.0075, 1.44) | 0.052 | 0.66 |
| TNM Stage (III versus | 2.69 | (0.91, 8.75) | 0.081 | 0.61 |
| II) | | | | |
| Age | 1.037 | (0.98, 1.097) | 0.18 | 0.60 |
| Sex (male versus female) | 1.97 | (0.68, 6.08) | 0.22 | 0.58 |
| Smoking status (Yes | 0.94 | (0.34, 2.59) | 0.91 | 0.51 |
| versus No) | | | | |

Multivariate Analysis:

In the logistic regression model including all the variables used to perform univariate analyses, we obtained an AUC of 0.84 and the covariates: age (p-value=0.0045), tumor size (p-value=0.022) and CCP score (p-value=0.026) were found to be statistically significant (Table 5). In the multivariate model with the covariates that reached significance in univariate analyses: CCP score, lymph-vascular invasion and nuclear grade, only lymph-vascular invasion (p-value=0.019) was statistically significant (Table 6) and the AUC associated to the model was 0.77. A step-wise variable selection was used to determine a subset of predictor variables (Table 7); the AUC was 0.84 and the following variables reached the significance level: age (p-value=0.0057), CCP score (p-value=0.0072), lymph-vascular invasion (p-value=0.022) and tumor size (p-value=0.047). We obtained an AUC of 0.78 when we excluded CCP score from the model (the relative predictive value of the model in terms of AUC increases by about 8% when the CCP score is added). There was no statistically significant quadratic or cubic effect of CCP score or any clinical variables.

Model Diagnostic:

The diagnostic plots for the univariate analyses indicated that the control with CCP score 1.04 is causing instability in the parameter estimate but not on the model fit. None of the diagnostic plots of the other univariate models indicated an influential observation.

Plots of the Pearson residuals and the deviance residuals indicated that one case was poorly accounted for by our 3 multivariate models. The index plots of DFBETAS indicated that the same observation was influential in the parameter estimates of those models. Another case was also an influential observation for the model obtained using step-wise variable selection.

TABLE 5
Multivariate logistic regression model including age,
tumor size, CCP score, lymph-vascular invasion sex,
stage, nuclear grade and smoking status as covariates.

| Variable | Odds ratio | 95% CI for OR | P-value |
|---|---|---|---|
| Age* | 1.22 | (1.04, 1.23) | 0.0045 |
| Tumor size | 1.34 | (1.06, 1.78) | 0.022 |
| CCP | 3.40 | (1.24, 11.27) | 0.026 |
| Lymph-vascular invasion | 3.13 | (0.68, 15.55) | 0.14 |
| (Yes versus No) | | | |
| Sex (male versus female) | 3.15 | (0.61, 2.85) | 0.19 |
| Stage (III versus II) | 2.48 | (0.49, 13.61) | 0.27 |
| Nuclear grade (high versus low) | 1.76 | (0.36, 8.78) | 0.47 |
| Smoking status (Yes versus No) | 0.65 | (0.14, 2.85) | 0.57 |

*The effect seen could be due to the bias associated with case-control selection.

TABLE 6
Multivariate logistic regression model including CCP score,
lymph-vascular invasion and nuclear grade as covariates.

| Variable | Odds ratio | 95% CI for OR | P-value |
|---|---|---|---|
| CCP | 2.14 | (0.09, 0.81) | 0.082 |
| Lymph-vascular invasion | 3.94 | (1.28, 13.03) | 0.019 |
| (Yes versus No) | | | |
| Nuclear grade (high versus low) | 1.99 | (0.56, 7.29) | 0.29 |

TABLE 7
Multivariate logistic regression model obtained
using step-wise variable selection: age, CCP score,
tumor size and lymph-vascular invasion are the covariates

| Variable | Odds ratio | 95% CI for OR | P-value |
|---|---|---|---|
| Age | 1.11 | (1.03, 1.19) | 0.0057 |
| CCP Tumor | 3.89 | (1.44, 10.47) | 0.0072 |
| size | 1.26 | (1.003, 1.56) | 0.047 |
| Lymph-vascular invasion (Yes | 4.46 | (1.24, 16.03) | 0.022 |
| versus No) | | | |

Exploratory Analysis:

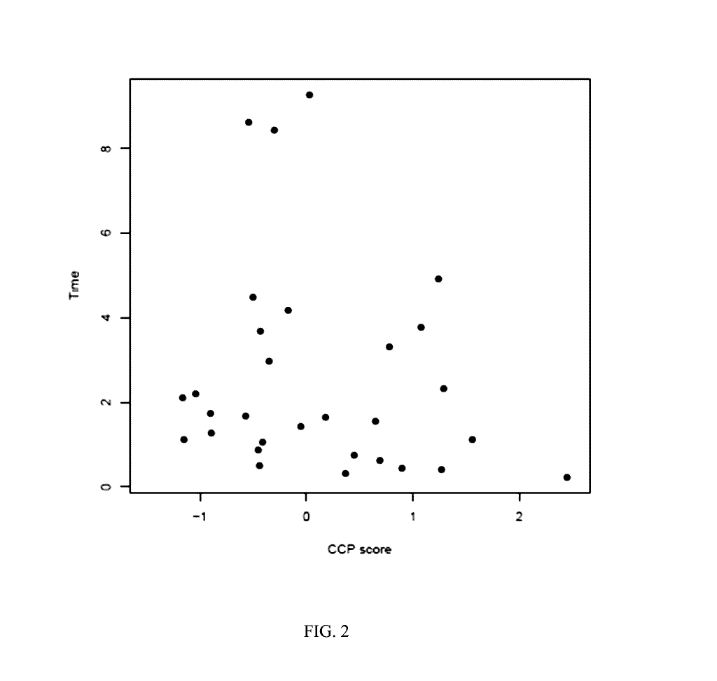

FIG. 2 provides the time versus CCP score for all patients with metastatic cancer. CCP scores tended to be higher for patients who showed early metastatic progression of the disease and lower for patients whose cancer metastasized later (FIG. 2).

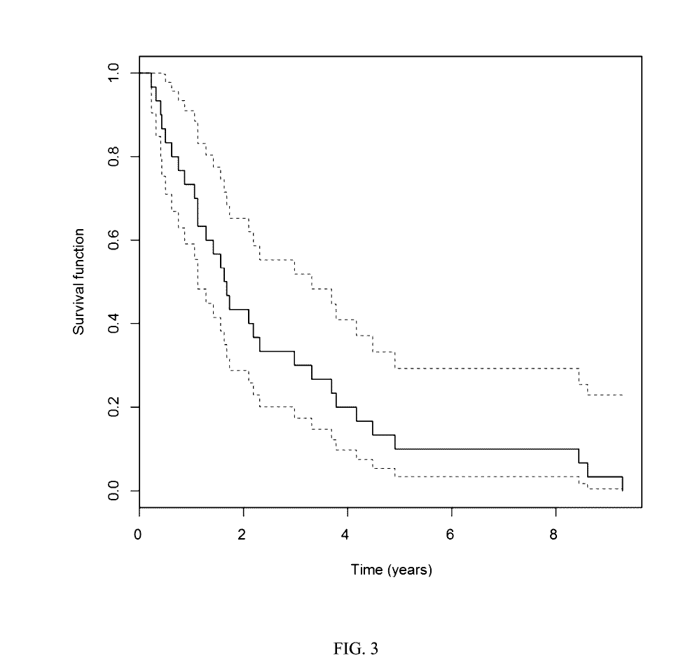

FIG. 3 provides the Kaplan-Meier estimate with 95% confidence bounds for all patients with metastatic cancers.

### CONCLUSION

The association between CCP score and metastatic progression of cancer in clear cell renal cell carcinoma was evaluated. It was found that CCP score by itself or after adjusting for other clinical variables significantly predicted risk of metastatic cancer.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference. The mere mentioning of the publications and patent applications does not necessarily constitute an admission that they are prior art to the instant application.

Although the foregoing invention has been described in some detail by way of illustration and example for purposes of clarity of understanding, it will be clear to one skilled in the art that certain changes and modifications may be practiced within the scope of the appended claims.

What is claimed is:

1. An in vitro method for diagnosing the prognosis of a test patient having renal cancer or the likelihood of renal cancer recurrence or metastatic progression in said test patient, comprising:
(1) measuring, in a sample obtained from said test patient, the expression levels of a panel of genes comprising at least 3 test genes selected from Table 1;
(2) providing a test expression score by (a) weighting the determined expression of each gene in said panel of genes with a predefined coefficient (which may be 0), and (b) combining the weighted expression of each gene in said panel of genes to provide said test expression score, wherein said test genes are weighted to contribute at least 25% to said test expression score; and
(3) diagnosing said test patient as having either (a) an increased likelihood of renal cancer recurrence or metastatic progression death based at least in part on said test expression score exceeding a first reference expression score or (b) no increased likelihood of renal cancer recurrence or metastatic progression based at least in part on said test expression score not exceeding a second reference expression score.

2. The method of claim 1, wherein said test genes are weighted to contribute at least 30%, 40%, 50%, 60%, 70%, 80%, 90%, 95%, 96%, 97%, 98%, 99%, or 100% of the total weight given to the expression of all of said panel of genes in said test expression score.

3. The method of either claim 1 or claim 2, wherein said panel of genes comprises at least 4, 5, 6, 7, 8, 9, 10, 12, 14, 16, 18, 20, 22, 24, 26, 28, 30 or 31 test genes selected from Table 1.

4. The method of any one of claims 1 to 3, wherein said measuring step comprises:
measuring the amount of panel mRNA in said sample transcribed from each of between 3 and 500 panel genes, or measuring the amount of cDNA reverse transcribed from said panel mRNA; and
measuring the amount of housekeeping mRNA in said sample transcribed from one or more housekeeping genes, or measuring the amount of cDNA reverse transcribed from said housekeeping mRNA.

5. The method of any one of claims 1 to 4, wherein said first and second reference expression scores are the same.

6. The method of any one of claims 1 to 5, wherein half of cancer patients in a reference population have an expression score exceeding said first reference expression score and half of cancer patients in said reference population have an expression score not exceeding said first reference expression score.

7. The method of any one of claims 1 to 4, wherein one third of cancer patients in a reference population have an expression score exceeding said first reference expression score and one third of cancer patients in said reference population have an expression score not exceeding said second reference expression score.

8. The method of claim 7, comprising diagnosing said test patient as having (a) an increased likelihood of renal cancer recurrence or metastatic progression if said test expression score exceeds said first reference expression score; (b) a decreased likelihood of renal cancer recurrence or metastatic progression if said test expression score does not exceed said second reference expression score; or (c) neither increased nor decreased (i.e., consistent) likelihood of renal cancer recurrence or metastatic progression if said test expression score exceeds said second reference expression score but does not exceed said first reference expression score.

9. The method of any one of claims 1 to 8, wherein renal cancer recurrence is chosen from the group consisting of distant metastasis of the primary cancer; local metastasis of the primary cancer; recurrence of the primary cancer; progression of the primary cancer; and development of locally advanced, metastatic disease.

10. A method for determining a renal cancer patient's likelihood of cancer recurrence or metastatic progression, comprising:
(1) measuring, in a sample obtained from said patient, the expression levels of a panel of genes comprising at least 3 test genes selected from Table 1;
(2) providing a test expression score by (1) weighting the determined expression of each gene in said panel of genes with a predefined coefficient (which may be 0), and (2) combining the weighted expression to provide said test expression score, wherein said test genes are weighted to contribute at least 25% to said test expression score;
(3) providing a test prognostic score combining said test expression score with at least one test clinical score representing at least one clinical variable; and
(4) diagnosing said patient as having either (a) an increased likelihood of cancer recurrence or cancer-specific death based at least in part on said test prognostic score exceeding a first reference prognostic score or (b) no increased likelihood of cancer recurrence or cancer-specific death based at least in part on said test prognostic score not exceeding a second reference prognostic.

11. The method of claim 10, wherein said at least one clinical score incorporates at least one clinical variable chosen from the group consisting of the size of the surgically-excised primary tumor, the Fuhrman nuclear grade of tumor cells, the stage of the tumor according to standard staging regimes, histological examination of the surgical margins, and evidence for lymph-vascular invasion.

12. An in vitro method of classifying renal cancer comprising:
(1) measuring the expression of a panel of genes comprising at least 3 genes from Table 1 in a sample;
(2) providing a test value by
(a) weighting the determined expression of each of a plurality of test genes selected from the panel of genes with a predefined coefficient, wherein said plurality of test genes comprises said at least 3 genes from Table 1; and
(b) combining the weighted expression to provide the test value, wherein the combined weight given to said at least 3 genes from Table 1 is at least 40% of the total weight given to the expression of said plurality of test genes; and
(3) correlating said test value to
(a) an unfavorable renal cancer classification if said test value is representative of high expression of the plurality of test genes; or
(b) a favorable renal cancer classification if said test value is representative of low or normal expression of the plurality of test genes.

13. The method of claim 12, wherein at least 75% of said plurality of test genes are genes from Table 1.

14. The method of claim 13, wherein said panel of genes and said plurality of test genes comprise at least 4, 5, 6, 7, 8, 9, 10, 12, 14, 16, 18, 20, 22, 24, 26, 28, 30 or 31 genes selected from Table 1.

15. The method of claim 12, wherein said unfavorable renal cancer classification is chosen from the group consisting of (a) a poor prognosis, (b) an increased likelihood of metastatic progression, (c) an increased likelihood of cancer recurrence, (d) an increased likelihood of cancer-specific death, or (e) a decreased likelihood of response to treatment with a particular regimen.

16. The method of claim 15, wherein said unfavorable cancer classification is an increased likelihood of cancer recurrence.

17. The method of claim 15, wherein said unfavorable renal cancer classification is an increased likelihood of metastatic progression.

18. The method of claim 12, wherein said favorable renal cancer classification is chosen from the group consisting of (a) a good prognosis, (b) no increased likelihood of metastatic progression, (c) no increased likelihood of cancer recurrence, (d) no increased likelihood of cancer-specific death, or (e) an increased likelihood of response to treatment with a particular regimen.

19. The method of claim 18, wherein said favorable cancer classification is no increased likelihood of cancer recurrence.

20. The method of claim 18, wherein said favorable cancer classification is no increased likelihood of metastatic progression.

21. A method of determining gene expression in a tumor sample, comprising:
(1) obtaining a tumor sample from a patient identified as having renal cancer;
(2) determining the expression levels of a panel of genes in said tumor sample including at least 3 genes chosen from Table 1; and
(3) providing a test value by (a) weighting the determined expression of each of a plurality of test genes selected from said panel of genes with a predefined coefficient, and (b) combining the weighted expression to provide said test value, wherein at least 75%, at least 85% or at least 95% of said plurality of test genes are genes chosen from Table 1.

22. The method of claim 21, wherein at least 90% of said plurality of test genes are gene chosen from Table 1.

23. The method of claim 21 or 22, wherein said determining step comprises:
measuring the amount of mRNA in said tumor sample transcribed from each of between 6 and 200 cell-cycle genes; and
measuring the amount of mRNA of one or more housekeeping genes in said tumor sample.

24. The method of claim 21 or 22 or 23, wherein the expression of at least 8 genes chosen from Table 1 are determined and weighted.

25. A method of prognosing renal cancer comprising:
(1) determining in a tumor sample from a patient identified as having renal cancer the expression of a panel of genes in said tumor sample including at least 4 cell-cycle genes;
(2) providing a test value by (1) weighting the determined expression of each of a plurality of test genes selected from said panel of genes with a predefined coefficient, and (2) combining the weighted expression to provide said test value, wherein at least 75%, at least 85% or at least 95% of said plurality of test genes are cell-cycle genes; and
(3) correlating an increased level of expression of said plurality of test genes to a poor prognosis.

26. The prognosis method of claim 25, further comprising comparing said test value to a reference value, and correlating to an increased likelihood of poor prognosis if said test value is greater than said reference value.

27. The prognosis method of claim 25, wherein the expression levels of from 6 to about 200 cell-cycle genes are measured.

28. The method of any one of claim 25 to 27, wherein said determining step comprises:
measuring the amount of mRNA of from 6 to about 200 cell-cycle genes in said tumor sample; and
measuring the amount of mRNA of one or more housekeeping genes in said tumor sample.

29. A diagnostic kit for prognosing cancer in a patient diagnosed as having renal cancer comprising, in a compartmentalized container:
(1) a plurality of PCR primer pairs for PCR amplification of at least 5 test genes, wherein less than 10%, 30% or less than 40% of all of said at least 8 test genes are non-cell-cycle genes; and
(2) one or more PCR primer pairs for PCR amplification of at least one housekeeping gene.

30. A diagnostic kit for prognosing cancer in a patient identified as having renal cancer, comprising, in a compartmentalized container:
(1) a plurality of probes for hybridizing to at least 5 test genes under stringent hybridization conditions, wherein less than 10%, 30% or less than 40% of all of said at least 8 test genes are non-cell-cycle genes; and
(2) one or more probes for hybridizing to at least one housekeeping gene.

31. The kit of claim 29 or 30, wherein cell-cycle genes constitute no less than 10% of the total number of said test genes.

32. The kit of any one of claims 29 to 31, wherein cell-cycle genes constitute no less than 20% of the total number of said test genes.

33. Use of
(1) a plurality of PCR primer pairs suitable for PCR amplification of at least 4 cell-cycle genes; and
(2) one or more PCR primer pairs suitable for PCR amplification of at least one housekeeping gene,
for the manufacture of a diagnostic product for determining the expression of said test genes in a tumor sample from a patient identified as having renal cancer to predict the prognosis of cancer, wherein an increased level of said expression indicates a poor prognosis or an increased likelihood of recurrence of cancer or metastatic progression in the patient.

34. The use of claim 33, wherein said plurality of PCR primer pairs are suitable for PCR amplification of at least 8 cell-cycle genes.

35. The use of claim 33 or 34, wherein said plurality of PCR primer pairs are suitable for PCR amplification of from 4 to about 300 test genes, no greater than 10%, 30% or less than 50% of which being non-cell-cycle genes.

36. The use of claim 33 or 34, wherein said plurality of PCR primer pairs are suitable for PCR amplification of from 20 to about 300 test genes, at least 25% of which being cell-cycle genes.

37. Use of
(1) a plurality of probes for hybridizing to at least 4 cell-cycle genes under stringent hybridization conditions; and
(2) one or more probes for hybridizing to at least one housekeeping gene under stringent hybridization conditions,
for the manufacture of a diagnostic product for determining the expression of said test genes in a tumor sample from a patient identified as having renal cancer to predict the prognosis of cancer, wherein an increased level of said expression indicates a poor prognosis or an increased likelihood of recurrence of cancer in the patient.

38. The use of claim 37, wherein said plurality of probes are suitable for hybridization to at least 8 different cell-cycle genes.

39. The use of claim 37 or 38, wherein said plurality of probes are suitable for hybridization to from 4 to about 300 test genes, no greater than 10%, 30% or less than 50% of which being non-cell-cycle genes.

40. The use of claim 37 or 38, wherein said plurality of probes are suitable for hybridization to from 20 to about 300 test genes, at least 25% of which being cell-cycle genes.

41. A system for prognosing renal cancer comprising:
(1) a sample analyzer for determining the expression levels of a panel of genes in said tumor sample including at least 4 cell-cycle genes, wherein the sample analyzer contains the tumor sample which is from a patient identified as having renal cancer, or cDNA molecules from mRNA expressed from the panel of genes; and
(2) a first computer program for (a) receiving gene expression data on at least 4 test genes selected from the panel of genes, (b) weighting the determined expression of each of the test genes, and (c) combining the weighted expression to provide a test value, wherein at least 50%, at least at least 75% of at least 4 test genes are cell-cycle genes; and
(3) a second computer program for comparing the test value to one or more reference values each associated with a predetermined degree of risk of cancer recurrence or metastatic progression.

42. The system of claim 41, further comprising a display module displaying the comparison between the test value to the one or more reference values, or displaying a result of the comparing step.

43. A method of treating renal cancer patients, comprising:
(1) measuring, in one or more patient samples, the expression levels of a panel of genes comprising at least 3 test genes selected from Table 1;
(2) providing a test expression score by (1) weighting the determined expression of each gene in said panel of genes with a predefined coefficient (which may be 0), and (2) combining the weighted expression to provide said test expression score, wherein said test genes are weighted to contribute at least 25% to said test expression score;
(3) optionally providing a test prognostic score combining said test expression score with at least one test clinical score representing at least one clinical variable; and
(4) recommending or prescribing or administering
(a) a treatment regimen comprising an anti-cancer drug and/or cytokine immunotherapy, antiangiogenic agents, and/or mTOR kinase inhibitors for a patient in whose sample said test expression score or test prognostic score exceeds a first reference expression or first reference prognostic score; or
(b) a treatment regimen not comprising an anti-cancer drug and/or cytokine immunotherapy, antiangiogenic agents, and/or mTOR kinase inhibitors for a patient in whose sample said test expression score or test prognostic score does not exceed a second reference expression or second reference prognostic score.

44. The method of claim 43, wherein said first and second expression or prognostic reference scores are the same.

45. The method of claim 43 or 44, wherein half of cancer patients in a reference population have an expression or prognostic score exceeding said first reference expression or prognostic score and half of cancer patients in said reference population have an expression or prognostic score not exceeding said first reference expression or prognostic score.

46. The method of claim 43, wherein one third of cancer patients in a reference population have an expression or prognostic score exceeding said first reference expression or prognostic score and one third of cancer patients in said reference population have an expression or prognostic score not exceeding said second reference expression or prognostic score.

47. The method of claim 46, comprising diagnosing said test patient as having (a) an increased likelihood of renal cancer recurrence or metastatic progression if said test expression or prognostic score exceeds said first reference expression or prognostic score; (b) a decreased likelihood of renal cancer recurrence or metastatic progression if said test expression or prognostic score does not exceed said second reference expression or prognostic score; or (c) neither increased nor decreased (i.e., consistent) likelihood of renal cancer recurrence or metastatic progression if said test expression or prognostic score exceeds said second reference expression or prognostic score but does not exceed said first reference expression or prognostic score.
